(19) United States
(12) Patent Application Publication
(10) Pub. No.: US 2025/0145578 A1
(43) Pub. Date: May 8, 2025

(54) SMALL MOLECULES TARGETTING EIF3E TO INHIBIT TUMOR GROWTH PROGRESSION, AND METASTASIS

(71) Applicant: The Regents of the University of Colorado, A Body Corporate, Aurora, CO (US)

(72) Inventors: Heide Ford, Aurora, CO (US); Rui Zhao, Aurora, CO (US); William Old, Aurora, CO (US); Xiang Wang, Aurora, CO (US)

(21) Appl. No.: 18/839,131

(22) PCT Filed: Feb. 21, 2023
(86) PCT No.: PCT/US2023/013474

§ 371 Date: Aug. 16, 2024

Related U.S. Application Data
(60) Provisional application No. 63/311,808, filed on Feb. 18, 2022.

(51) Int. Cl. C07D 295/26; A61K 31/40; A61K 31/4453; A61K 31/454; A61K 31/496; A61K 31/5377; C07D 205/04; C07D 207/00; C07D 231/12; C07D 233/61; C07D 401/04

(52) U.S. Cl. CPC C07D 295/26; A61K 31/40; A61K 31/4453; A61K 31/454; A61K 31/496; A61K 31/5377; C07D 205/04; C07D 207/00; C07D 231/12; C07D 233/61; C07D 401/04

(57) ABSTRACT

The disclosure includes a method of inhibiting a eukaryotic translation initiation factor 3e (eIF3e) comprising contacting the eIF3e with a compound or salt of the Formula I, where the variables, e.g., Y, Z, R, and R¹R¹¹ are defined herein. The disclosure includes a method of modulating SIX1 and/or EYA2 levels or inhibiting SIX1/EYA2 interactions comprising contacting eukaryotic translation initiation factor 3e (eIF3e) with a compound of the formula. The disclosure includes a method of treating metastatic breast cancer (of all subtypes), glioblastoma, Wilms' tumor, ovarian cancer, lung cancer, cervical, oral cancer, or any cancer shown to be dependent on SIX1 or EYA proteins, which are regulated by eIF3e, in a patient comprising administering the patient a compound of the Formula I.

### CROSS REFERENCE TO RELATED APPLICATIONS

The application claims priority to U.S. Provisional Appl. No. 63/311,808 filed Feb. 18, 2022, which is hereby incorporated by reference in its entirety.

### STATEMENT OF GOVERNMENT SUPPORT

This invention was made in part with support from U.S. grant number CA224867. The U.S. government may have certain rights in this invention.

### BACKGROUND

Globally, the most common cancer in women is breast cancer (BC), with over 2 million cases and almost 685,000 deaths in 2020. Worldwide, 12% of all cancers diagnosed in 2020 were BC, and it is now the most commonly diagnosed cancer overall (having recently surpassed lung cancer). The post-treatment five-year survival rate for patients with a primary breast tumor is now close to 90%. Virtually all BC deaths result from metastatic disease rather than from the primary tumor. Stage IV or metastatic breast cancer (MBC) is breast cancer that has spread to the other organs, including brain, liver, bones, and lungs. There are 150,000 women in the U.S. living with metastatic breast cancer. Treatments for MBC have gradually advanced, with 5-year survival improving from 10% in 1985 to 27% today. Still, median survival is just three years. In 2021, 43,600 U.S. women are expected to die from MBC.

In general, MBC treatments are either non-specific, affecting cancerous and healthy rapidly dividing cells causing severe and sometimes fatal side effects, or too specific, only effective for a limited number (10-30%) of patients. For example, the best-selling breast cancer drug in the U.S. is Herceptin, a monoclonal antibody that specifically targets the HER2 receptor which is overexpressed in 15%-20% of breast cancer patients and Herceptin is only effective in 30% of this small subset. Despite the limited patient population, rapid loss of efficacy, and associated toxicity, Herceptin sales in 2019 were $6 billion, an indication of the dire need for MBC treatments. New agents are critically needed that are 1) highly specific to MBC, 2) broadly potent among MBC stages and types, and 3) address different pathways and have low toxicity, so that they can be combined with other agents to prevent the emergence of drug resistance. Most non-specific cancer therapeutics available today meet only the second criterion, often with dangerous toxicities. Most targeted MBC therapeutics may meet the first and third criteria but address only small patient populations.

Eukaryotic translation initiation factor 3e (eIF3e) is a factor known to be critical for the translation of a subset of mRNAs that regulate key processes such as responses to stress, epithelial to mesenchymal transition, proliferation, and survival, and that is known to be dysregulated in numerous cancers, including breast cancer. For example, we have shown that eIF3e affects cellular levels of both SIX1 and EYA2, two transcription factors that act in a complex to enhance breast cancer metastasis. We have shown that eIF3e also regulates stress response pathways by, for example, regulating cellular levels of the following: c-Jun that responds to the stress of glucose deprivation, transcription factor HIF1 that is upregulated in response to hypoxia, and oxidative phosphorylation. Small molecules that inhibit eIF3e are desirable in order to inhibit metastasis by, among other things, reducing stress responses that allow metastatic cells to thrive in new microenvironments and reducing SIX1/EYA2 protein levels and SIX1/EYA2 interactions within cancer cells. This disclosure meets the need for small molecule eIF3e inhibitors and provides additional advantages, discussed herein.

### SUMMARY

The disclosure provides compounds and pharmaceutically acceptable salts of Formula I

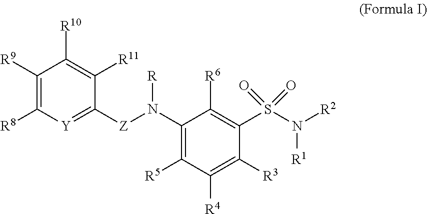

Certain compounds of Formula I are useful as direct inhibitors of eIF3e.

Within Formula I the variables, e.g., R, R¹-R¹¹, Y and Z, carry the following definitions.
Y is N or CR⁷;
Z is methylene (—CH₂—), keto (—(C═O)—), or is taken together with R⁵ to form a 5-membered partially unsaturated ring that contains 1 or two nitrogen atoms;
R is hydrogen or methyl;
R¹ is C₁-C₆alkyl or (C₃-C₆cyclopropyl)C₀-C₂alkyl; and
R² is hydrogen or C₁-C₆alkyl; or
R¹ and R² are taken together to form a 5- or 6-membered heterocycloalkyl optionally containing one additional heteroatom selected from N, O, and S, and optionally substituted with one or two substituents independently chosen from halogen, hydroxyl, C₁-C₄alkyl, C₁-C₄alkoxy, C₁-C₂haloalkyl, and C₁-C₂haloalkoxy;
R³ is hydrogen, halogen, hydroxyl, C₁-C₆alkyl, C₁-C₆alkoxy, C₁-C₂haloalkyl, C₁-C₂haloalkoxy, or (C₃-C₆cycloalkyl)C₀-C₂alkyl-O—;
R⁴, R⁶, R⁷, and R¹¹ are independently hydrogen, halogen, hydroxyl, C₁-C₄alkyl, C₁-C₄alkoxy, C₁-C₂haloalkyl, and C₁-C₂haloalkoxy;
R⁵ is hydrogen, halogen, hydroxyl, C₁-C₄alkyl, C₁-C₄alkoxy, C₁-C₂haloalkyl, or C₁-C₂haloalkoxy or R⁵ is taken together with Z to form a partially unsaturated ring that contains 1 or 2 nitrogen atoms;
one of R⁸ and R⁹ is halogen, nitro, phenyl, di-C₁-C₄alkylamino, or a 4-6 membered saturated, partially unsaturated or aromatic heterocyclic group containing 1 or 2 additional heteroatoms selected from N, O, and S, each of which is optionally substituted with one or more substituents independently chosen from halogen, hydroxyl, cyano, amino, C₁-C₄alkyl, C₁-C₄alkoxy, C₁-C₂haloalkyl, and C₁-C₂haloalkoxy, and the other of R⁸ and R⁹ is hydrogen, halogen, hydroxyl, cyano, amino, C₁-C₂alkyl, C₁-C₂alkoxy, C₁-C₂haloalkyl, and C₁-C₂haloalkoxy;
R¹⁰ is hydrogen, halogen hydroxyl, cyano, amino, C₁-C₆alkyl, C₁-C₆alkoxy, mono- or di-C₁-C₆alkylamino, mono- or di-alkylamide, C₁-C₂haloalkyl, and C₁-C₂haloalkoxy where any alkyl or alkoxy at R¹, R², R³, or R¹⁰ optionally has one methylene of the alkyl chain replaced by O, NH, N(C₁-C₄alkyl), or S.

When claimed solely as compounds rather than for use in a method, it is required that R³ is other than hydrogen, and that several previously reported compounds (listed in the Detailed Description section and in the Claims) are excluded.

The disclosure further provides pharmaceutical compositions and methods of treatment.

The disclosure includes a method of inhibiting a eukaryotic translation initiation factor 3e (eIF3e) comprising contacting the eIF3e with a compound or salt of the Formula I.

The disclosure includes a method of modulating SIX1 and/or EYA2 levels or inhibiting SIX1/EYA2 interactions comprising contacting eukaryotic translation initiation factor 3e (eIF3e) with a compound of the formula.

The disclosure includes a method of treating metastatic breast cancer (of all subtypes), glioblastoma, Wilms' tumor, ovarian cancer, lung cancer, cervical, oral cancer, or any cancer shown to be dependent on SIX1 or EYA proteins, which are regulated by eIF3e, in a patient comprising administering the patient a compound of the Formula I.

### BRIEF DESCRIPTION OF THE DRAWINGS

FIG. 1. eIF3e amplification and expression correlates with worse prognosis in breast cancer patients. FIG. 1A. Proportion of breast cancer patients with eIF3e amplification in the METABRIC 2016 and TCGA 2015 datasets. FIG. 1B. Overall survival in breast cancer patients with/without eIF3e amplification from METABRIC 2016 and TCGA 2015 datasets. FIG. 1C. Meta-analysis of relapse-free survival in breast cancer patients with the highest 50% of eIF3e expression compared to the lowest 50% eIF3e expression from Kaplan-Meier plotter. The low and high numbers below the graph are number of patients in each group. The gray trace represents the high expression group and the black trace the low expression group.

FIG. 2. eIF3e is significantly stabilized with 8430 treatment. FIG. 2A. ITSAs were performed by mass spectrometry of Vehicle (DMSO) or 20 μM 8430 treated samples to identify proteins more stable in solution with 8430 treatment compared to DMSO. FIG. 2B. Purified eIF3e is stabilized by 20 μM 8430 and its analog (compound 12) as demonstrated by Western blot of protein remained in soluble fraction at indicated temperatures.

FIG. 3. 8430 treatment of breast cancer cells moderately affects global translation. Cells were treated overnight with 20 μM 8430 followed by incorporation of O-propargyl-puromycin (OPP) into newly synthesized proteins then fluorescently labeled using Click-It Plus OPP Alexa Fluor 488 Protein Synthesis Assay Kit. Fluorescent intensity at 488 nm divided by DAPI fluorescent intensity was used to determine relative levels of translation. *p≤0.05; **p≤0.01.

FIG. 4. Glucose deprivation of HEK293T cells results in enhanced c-Jun protein levels, both 8430 and eIF3e KD reverse this induction. FIG. 4A. Varying doses of 8430 and effects on both SIX1 and c-Jun in glucose depleted media. FIG. 4B. Effects of 20 μM 8430 and KD of eIF3e on c-Jun protein levels. HEK293T cells were used as these cells were first shown to exhibit eIF3-mediated changes in c-Jun in response to glucose deprivation.

FIG. 5. Transient KD of eIF3e in MCF7-SIX1 cells using siRNA demonstrates a reduction in both EYA2 and SIX1 levels, similar to what is observed in MCF7-SIX1 cells treated with 8430.

FIG. 6. Transient KD of eiF3e phenocopies 8430 treatment in MCF7-Six1 cells. eiF3e KD performed with smart pools and 8430 was used at 20 μM, after which Incucyte® live cell imaging was used to monitor confluency over time. ***p≤0.001.

FIG. 7. Compound 8430 treatment of ER+ breast cancer cells overexpressing SIX1 (FIG. 7A) inhibits their growth, whereas it has no effect on the growth of immortalized but otherwise normal (non-transformed) MCF10A cells mammary epithelial cells (FIG. 7B). Growth measured using Incucyte live cell imaging. ****p≤0.0001.

FIG. 8. The effect of 8430 and a subset of analogs (all at 10 μM) on MCF7-SIX1 cell growth as monitored using Incucyte live cell imaging. Representative data is shown of three separate experiments performed with 8430 and a subset of the 20 analogs. FIG. 8A. Growth of cells treated with DMSO, 8430, compound 3, or compound 6. FIG. 8B. Growth of cells treated with DMSO, 8430, compound 9, or compound 10, or compound 12. FIG. 8C. Growth of cells treated with DMSO, 8430, compound 13 or compound 17.

FIG. 9. Compound 12 reduces SIX1 and EYA2 levels. MCF7-Six1 cells were treated with analog 12 for 48 hours after which cells were lysed and Western blot performed. C12 phenocopies 8430 at a 4-fold lower dose.

FIG. 10A. HEK293T cells were glucose starved for 48 hours+/−8430 (20 μM) or C12 (5 μM) treatment followed by WB analysis. FIG. 10B. MCF7-SIX1 cells were treated overnight+/−8430 (20 μM) or C12 (5 μM) then placed in a hypoxic chamber (1% O2) for four hours followed by WB analysis. FIG. 10C. Oxygen consumption rate (OCR)+/−8430 (20 μM) or C12 (10 μM) treatment. Mitochondrial stress test conditions: Oligomycin 2.5 μM, FCCP 2 μM, Rotenone/Antimycin A 0.5 μM each. FIG. 10D. ATP production calculated as (Last rate measurement before oligomycin injection)−(minimum rate measurement after oligomycin injection). *p≤0.05, ****p≤0.0001.

FIG. 11. Incucyte growth assays demonstrate that both eIF3e KD and 8430 treatment can inhibit the growth of an ER+ patient derived xenograft (PDX) breast cancer line (UCD4).

FIG. 12. Cell-titer assay for cancer cells treated with DMSO or compounds of the disclosure. FIG. 12A. Cell viability as measured by RLU for Normal but immortalized T47D cells treated with DMSO or 10 μM compound 8430, 30, 31, 32, 9, and 33 for 70 hrs. ; FIG. 12B. RLU for breast cancer T47D cells treated with DMSO or 10 μM compound 8430, 30, 31, 9, 32, and 33 for 70 hrs. FIG. 12C. RLU for breast cancer MCFsix1 cells treated with DMSO or 10 μM compound 8430, 30, 31, 9, and 33 for 70 hrs.

FIG. 13. Quantitation of IP—WB shows decrease in eIF3D levels in the complex with 8430 treatment.

FIG. 14. Western blot showing eIF3e mediated translation of HIF1-alpha is inhibited via 8430 treatment.

FIG. 15. Primary tumor volume of MCFSIX1 tumor in animals treated with IP injected DMSO and 8430. 8430 inhibits primary tumor growth. It was originally thought that 8430 would only inhibit metastasis. See Zhou, et a., Cancer Research, (2020) 80(12): 2689-2702, but more recent data show it also inhibits primary tumor growth.

### DETAILED DESCRIPTION

### Terminology

In the description and claims, terms will carry the definitions set forth in this section unless the stated otherwise or contrary to the context. Unless defined otherwise, all technical and scientific terms used herein have the commonly understood by one of ordinary skill in the art to which the disclosure pertains. Although any methods and materials similar or equivalent to those described herein may be useful in the practice or testing of the embodiments of this disclosure; preferred methods and materials are described below.

The terms “a” and “an” do not denote a limitation of quantity, but rather denote the presence of at least one of the referenced items. The term “or” means “and/or”. The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50% means in the range of 45%-55%.

Numerical ranges recited herein by endpoints include all numbers and fractions subsumed within that range (e.g., 1 to 5 includes 1, 1.5, 2, 2.75, 3, 3.90, 4, and 5).

As used herein, the terms “administration of” and or “administering” a compound should be understood to mean providing a compound of the disclosure to a subject in need of treatment. “Administering” includes administration of a compound of the disclosure by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal means.

“Alleviating a disease or disorder symptom,” means reducing the severity of the symptom or the frequency with which such a symptom is experienced by a subject, or both.

A “Compound of Formula I” as used herein, refers to any compound within the scope of Formula I and, unless the context indicates otherwise, includes the pharmaceutically acceptable salts of Formula I. A Compound of Formula I encompasses a Compound of Formula I and its pharmaceutically acceptable salts.

The terms “comprises,” “comprising,” and the alternate transitional phrases “includes,” “including,” “contain,” and “containing” are open ended transitional phrases having the meaning ascribed to them in U.S. Patent Law. “Comprises” and the other open-ended terms encompass the intermediate term “consisting essentially of” and the closed ended terms “consisting of” and “consists of.” Claims reciting one of the open-ended transitional phrases can be written with any other transitional phrase, which may be more limiting, unless clearly precluded by the context or art.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary.

As used in the specification and the appended claims, the terms “for example,” “for instance,” “such as,” “including” and the like are meant to introduce examples that further clarify more general subject matter. Unless otherwise specified, these examples are provided only as an aid for understanding the disclosure and are not meant to be limiting in any fashion.

The terms “formula” and “structure” are used interchangeably herein.

The term “inhibit,” as used herein, refers to the ability of a compound of the disclosure to reduce or impede a described function, such as having inhibitory sodium channel activity. Preferably, inhibition is by at least 10%, more preferably by at least 25%, even more preferably by at least 50%, and most preferably, the function is inhibited by at least 75%. The terms “inhibit”, “reduce”, and “block” are used interchangeably herein.

The term “pharmaceutical composition” shall mean a composition comprising at least one active ingredient and a pharmaceutically acceptable carrier, such as a pharmaceutically acceptable excipient.

A “pharmaceutically acceptable excipient” means an excipient that is useful in preparing a pharmaceutical composition/combination that is generally safe, non-toxic and neither biologically nor otherwise undesirable, and includes an excipient that is acceptable for veterinary use as well as human pharmaceutical use. The term also encompasses any of the inactive agents approved for use pharmaceutical compositions in by a regulatory agency of the US Federal government or listed in the US Pharmacopeia for use in animals, including humans.

The term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

“Pharmaceutically acceptable” means physiologically tolerable, for either human or veterinary application. As used herein, “pharmaceutical compositions” include formulations for human and veterinary use.

The term “prevent,” as used herein, means to stop something from happening or to significantly reduce the likelihood of something happening, such as by taking advance measures against something possible or probable outcome. In the context of medicine, “prevention” includes an action taken to decrease the chance of getting a disease or condition.

A “subject” of analysis, diagnosis, or treatment is an animal. Such animals include mammals, preferably a human. As used herein, a “subject in need thereof” is a patient, animal, mammal, or human, who will benefit from the method of this disclosure.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, “treat,” “treating”, or “treatment” includes treating, ameliorating, or inhibiting an injury or disease related condition or a symptom of an injury or disease related condition. In one embodiment the disease, injury or disease related condition or a symptom of an injury or disease related condition is prevented, while another embodiment provides prophylactic treatment of the injury or disease related condition or a symptom of an injury or disease related condition. Prevention does not mean reducing to zero the probability that a disease or condition will occur. Rather, prevention means significantly reducing the probability that a disease or condition will occur in a patient or subject at risk for developing the disease or condition.

### Chemical Definitions

“Alkyl” is a branched or straight chain saturated aliphatic hydrocarbon group, having the specified number of carbon atoms, generally from 1 to about 8 carbon atoms. The term C₁-C₆-alkyl as used herein indicates an alkyl group having from 1, 2, 3, 4, 5, or 6 carbon atoms. Other embodiments include alkyl groups having from 1 to 6 carbon atoms, 1 to 4 carbon atoms or 1 or 2 carbon atoms, e.g., C₁-C₈-alkyl, C₁-C₄-alkyl, and C₁-C₂-alkyl. Examples of alkyl include, but are not limited to, methyl, ethyl, n-propyl, isopropyl, n-butyl, 3-methylbutyl, t-butyl, n-pentyl, sec-pentyl, heptyl, and octyl. “C₀-Cₙ alkyl” is used together with another group, e.g., C₀-C₄alkyl(C₃-C₇cycloalkyl), to indicate the other group, in this case C₃-C₇cycloalkyl, is bound to the group it substitutes either by a single covalent bond (C₀) or attached through an alkylene linker having the indicated number of carbon atoms.

“Alkoxy” is an alkyl group as defined above with the indicated number of carbon atoms covalently bound to the group it substitutes by an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

“Alkylamino” is an alkyl group as defined herein covalently bound to the group it substitutes by an amino linkage. An alkylamino group can be a mono-alkyl group in which the amino is a secondary amino (alkyl)NH—) or a di-alkyl group in which the amino is a tertiary amino, (alkyl1)(alkyl2)N—. The alkyl groups of a di-alkylamino are the same or different.

“Alkyl amide” is an alkyl group as defined herein covalently bound to the group it substitutes by an amide linkage. The amide linkage may be in either orientation, e.g., a group of the formula —NHC(O)-alkyl or a group of the formula —C(O)NH-alkyl.

“Cycloalkyl” is a saturated hydrocarbon ring group, having the specified number of carbon atoms. Monocyclic cycloalkyl groups typically+ have from 3 to about 8 carbon ring atoms, from 3 to 7 ring atoms, or from 3 to 6 (3, 4, 5, or 6) carbon ring atoms. Cycloalkyl substituents may be pendant from a substituted nitrogen, oxygen, or carbon atom, or a substituted carbon atom that may have two substituents may have a cycloalkyl group, which is attached as a spiro group. Examples of cycloalkyl groups include cyclopropyl, cyclobutyl, cyclopentyl, and cyclohexyl.

“Halogen” or “halo” includes bromo, chloro, fluoro, and iodo.

“Haloalkyl” indicates both branched and straight-chain alkyl groups having the specified number of carbon atoms, substituted with 1 or more halogen atoms, up to the maximum allowable number of halogen atoms. Examples of haloalkyl include, but are not limited to, trifluoromethyl, difluoromethyl, 2-fluoroethyl, 2,2,2-trifluoroethyl, and penta-fluoroethyl.

“Haloalkoxy” indicates a haloalkyl group as defined herein attached through an oxygen bridge (oxygen of an alcohol radical).

“Heterocyclic” is a ring or ring system having one or two saturated, unsaturated, or aromatic rings with at least one ring containing one, two, three, or four heteroatoms independently chosen from N, O, and S with remaining ring atoms being carbon. It is preferred that the total number of heteroatoms in a heterocyclic ring system is not more than 4 and that the total number of S and O atoms in a heteroaryl ring system is not more than 2. Monocyclic heteroaryl groups typically have from 5 to 7 ring atoms. In some embodiments bicyclic heteroaryl groups are 9- to 10-membered heteroaryl groups, that is, groups containing 9 or 10 ring atoms in which one 5- to 7-member aromatic ring is fused to a second aromatic or non-aromatic ring. When the total number of S and O atoms in an aromatic ring of the heteroaryl group exceeds 1, these heteroatoms are not adjacent to one another. Examples of heteroaryl groups include, but are not limited to, oxazolyl, pyrazyl, pyrazinyl, pyrazolopyrimidinyl, pyrazolyl, pyridazinyl, pyridyl, pyrimidinyl, pyrrolyl, quinolinyl, tetrazolyl, thiazolyl, thienyl pyrazolyl, morphonlinyl, thiophenyl, triazolyl, benzo[d]oxazolyl, benzofuranyl, benzothiazolyl, benzothiophenyl, benzoxadiazolyl, dihydrobenzodioxynyl, furanyl, imidazolyl, indolyl, and isoxazolyl.

“Heterocycloalkyl” is a saturated cyclic group containing 1 or more ring atoms independently chosen from N, O, and S with remaining ring atoms being carbon. Examples of heterocycloalkyls include tetrahydropyranyl, tetrahydrofuranyl, piperidinyl, piperazinyl, morpholinyl, thiomorpholinyl, oxazolidinyl, tetrahydrothiophenyl, tetrahydrothiopyranyl, thiazolidinyl, and pyrrolidinyl.

“Pharmaceutically acceptable salts” includes derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Alkali metal (for example, sodium, potassium or lithium) or alkaline earth metal (for example calcium) salts of organic (e.g., carboxylic) acids can also be made.

Examples of pharmaceutically acceptable salts include, but are not limited to, mineral or organic acid salts of basic residues such as amines or nitrogen-containing heteroaryl rings (e.g., pyridine, quinoline, isoquinoline); alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, phosphoric, nitric and the like; and the salts prepared from organic acids such as acetic, propionic, succinic, glycolic, stearic, lactic, malic, malonic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, succinic, toluenesulfonic, methanesulfonic, ethane disulfonic, oxalic, α-ketoglutarate, α-glycerophosphate, isethionic, HO₂C—(CH₂)ₙ—CO₂H where n is 0-4, and the like.

Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, Nalkylglucamines, theobromine, purines, piperazine, piperidine, morpholine, Nethylpiperidine, and the like. It should also be understood that other carboxylic acid derivatives would be useful in the practice of this disclosure, for example, carboxylic acid amides, including carboxamides, lower alkyl carboxamides, dialkyl carboxamides, and the like.

Lists of additional suitable salts may be found, e.g., in G. Steffen Paulekuhn, et al., Journal of Medicinal Chemistry 2007, 50, 6665 and Handbook of Pharmaceutical Salts: Properties, Selection and Use, P. Heinrich Stahl and Camille G. Wermuth Editors, Wiley-VCH, 2002.

The term “substituted” means that any one or more hydrogens on the designated atom or group is replaced with a selection from the indicated group, provided that the designated atom's normal valence is not exceeded. Unless otherwise specified, each substituent is selected independently of other substituents. “Optionally substituted” means that 0 to the maximum allowable number of substituents are present. When the substituent is oxo (i.e., ═O) then 2 hydrogens on the atom are replaced. When an oxo group substitutes a heteroaromatic moiety, the resulting molecule can sometimes adopt tautomeric forms. For example, a pyridyl group substituted by oxo at the 2- or 4-position can sometimes be written as a pyridine or hydroxypyridine. Combinations of substituents and/or variables are permissible only if such combinations result in stable compounds or useful synthetic intermediates. A stable compound or stable structure is meant to imply a compound that is sufficiently robust to survive isolation from a reaction mixture and subsequent formulation into an effective therapeutic agent. Unless otherwise specified, substituents are named into the core structure. For example, it is to be understood that aminoalkyl means the point of attachment of this substituent to the core structure is in the alkyl portion and alkylamino means the point of attachment is a bond to the nitrogen of the amino group. However, a dash (“-”) indicates a point of attachment for a substituent. —C₁₋C₄alkyl(cycloalkyl) is attached at the 1 to 4 carbon alkylene linker.

Certain compounds of the disclosure may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers and mixtures thereof are encompassed. In these situations, single enantiomers, i.e., optically active forms, can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example using a chiral HPLC column. In addition, compounds with carbon-carbon double bonds may occur in Z- and E-forms, with all isomeric forms of the compounds being included in the present disclosure.

The disclosure includes deuterated compounds of Formula I in which any hydrogen is replaced by a deuterium. “Deuterated” mean that a hydrogen at the specified position is replaced by deuterium. In any sample of a compound of Formula I in which a position is deuterated some discrete molecules of the compound of Formula I will likely have hydrogen, rather than deuterium, at the specified position. However, the percent of molecules in the sample which have deuterium at the specified position will be much greater than would naturally occur. The deuterium at the deuterated position is enriched. The term “enriched” as used herein, refers to the percentage of deuterium versus other hydrogen species at that location. As an example, if it is said that a position in the compound of Formula I contains 50% deuterium enrichment, that means that rather than hydrogen at the specified position the deuterium content is 50%. For clarity, it is confirmed that the term “enriched” as used herein does not mean percentage enriched over natural abundance. In one embodiment, deuterated compounds of Formula A and Formula I will have at least 10% deuterium enrichment at any deuterated position. In other embodiments, there will be at least 50%, at least 90%, or at least 95% deuterium enrichment at the specified deuterated position or positions. A “deuterated substituent” is a substituent in which at least one hydrogen is replaced by deuterium at the specified percent enrichment.-“Optionally deuterated” means that the position may be at either hydrogen and the amount of deuterium at the position is only the naturally occurring level of deuterium or the position is enriched with deuterium above the naturally occurring deuterium level.

### Chemical Description

The disclosure includes compounds and pharmaceutically acceptable salts of the Formula I as delineated in the SUMMARY section. Additionally, the disclosure includes compounds and pharmaceutically acceptable salts of Formula I in which the variables, e.g., R, R¹-R¹¹, Y, and Z, have the following definitions. The disclosure includes any combination of the following variable definitions so long as a stable compound results.

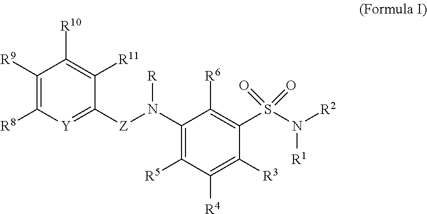

(i) R¹ and R² are taken together to form a 6-membered heterocycloalkyl optionally containing one additional heteroatom selected from N, O, and S, and optionally substituted with one or two substituents independently chosen from halogen, hydroxyl, C₁-C₄alkyl, C₁-C₄alkoxy, C₁-C₂haloalkyl, and C₁-C₂haloalkoxy.
(ii) R¹ and R² are taken together to form a piperazine, piperidine, or morpholine ring that is optionally substituted with one or two methyl substituents.
(iii) R¹ and R² are both methyl or both ethyl.
(iv) R³ is halogen, hydroxyl, methoxy, ethoxy, or cycloalkyloxy; and R⁴, R⁵, and R⁶ are all hydrogen.
(v) Z is keto.
(vi) Y is N.
(vii) Y is CR⁷ and R⁷ is hydrogen or methyl.
(viii) R⁹, R¹⁰, and R¹¹ are all hydrogen.
(ix) R⁸ is phenyl, di-methylamino, or a 4-, 5-, or 6-membered heterocycloalkyl ring optionally containing one additional heteroatom selected from N, O, and S, and optionally substituted with methyl or R⁸ is phenyl or a 5-membered heteroaryl ring optionally containing one or two additional heteroatoms selected from N, O, and S and optionally substituted with halogen, C₁-C₂alkyl, C₁-C₂alkoxy, trifluoromethyl, and trifluoromethoxy; and R⁹ is hydrogen or methyl.
(x) R⁸ is hydrogen or methyl; and
R⁹ is phenyl, di-methylamino, or a 4-, 5-, or 6-membered heterocycloalkyl ring optionally containing one additional heteroatom selected from N, O, and S, and optionally substituted with methyl or R⁸ is phenyl or a 5-membered heteroaryl ring optionally containing one or two additional heteroatoms selected from N, O, and S and optionally substituted with halogen, C₁-C₂alkyl, C₁-C₂alkoxy, trifluoromethyl, and trifluoromethoxy; and R⁹ is hydrogen or methyl.
(xi) R¹⁰ is hydrogen, halogen, C₁-C₂alkyl, C₁-C₂alkoxy, or dimethylamino-ethylcarboxamide.
(xii) R¹ and R² are both methyl or ethyl, or
R¹ and R² are taken together to form a morpholine, piperazine, or piperidine ring, each of which is optionally substituted with one or two methyl substituents.
R³ is halogen, hydroxyl, methoxy, or cyclopropyloxy;
R⁴, R⁵, and R⁶ are all hydrogen;
R⁷ and R¹¹ are both hydrogen or methyl;
One R⁸ and R⁹ is hydrogen or methyl; and
the other of R⁸ and R⁹ is a 4-, 5-, or 6-membered heterocycloalkyl ring optionally containing one additional heteroatom selected from N, O, and S, and optionally substituted with methyl or R⁸ is phenyl or a 5-membered heteroaryl ring optionally containing one or two additional heteroatoms selected from N, O, and S and optionally substituted with halogen, C₁-C₂alkyl, C₁-C₂alkoxy, trifluoromethyl, and trifluoromethoxy; and R⁹ is hydrogen or methyl; and
R¹⁰ is hydrogen, halogen, C₁-C₂alkyl, C₁-C₂alkoxy, or dimethylaminoethylcarboxamide.

Exemplified compounds of Formula I include:

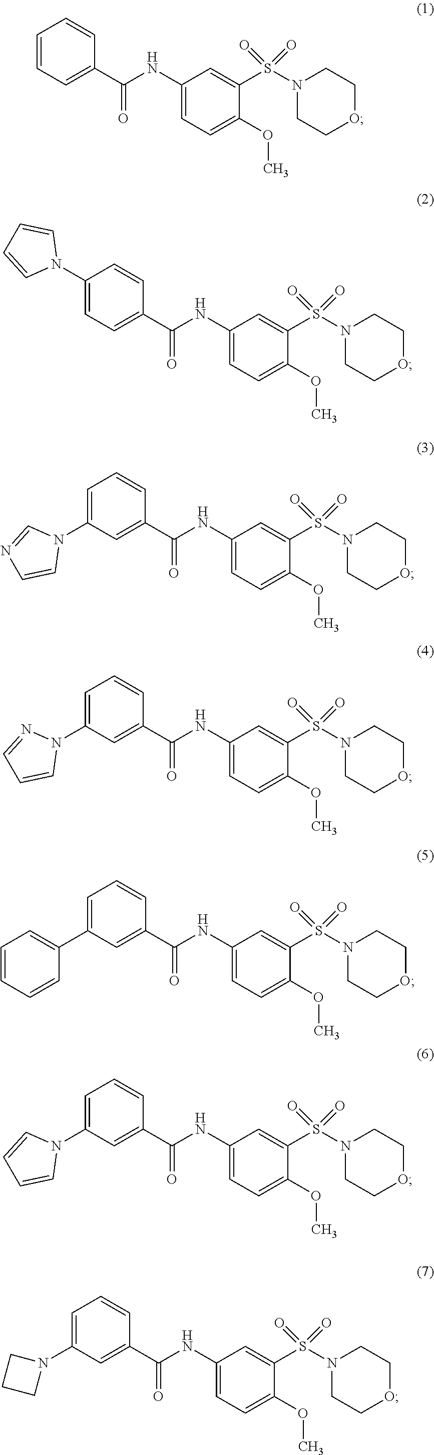

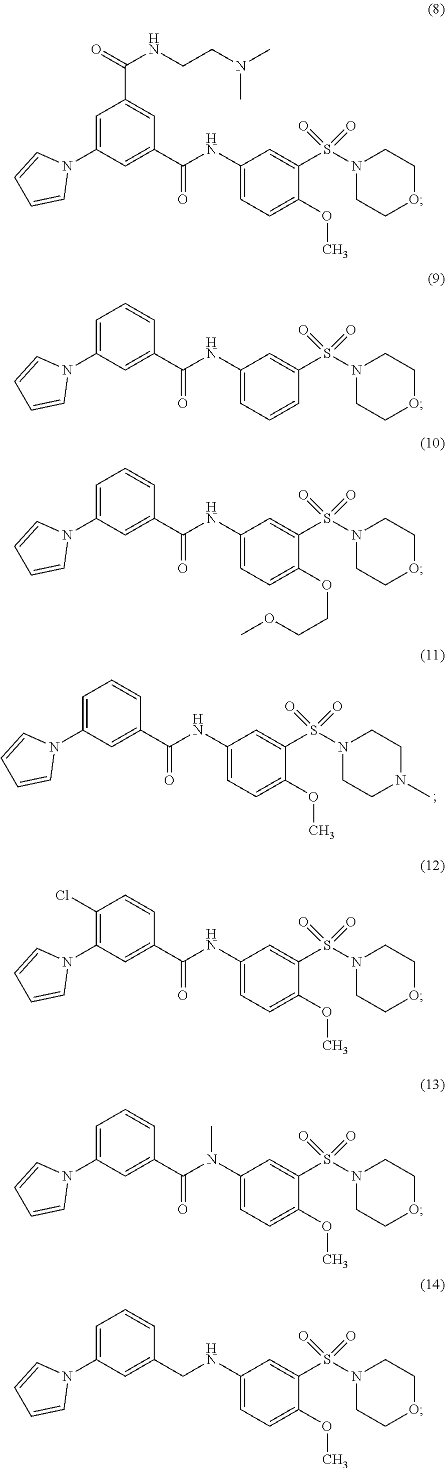

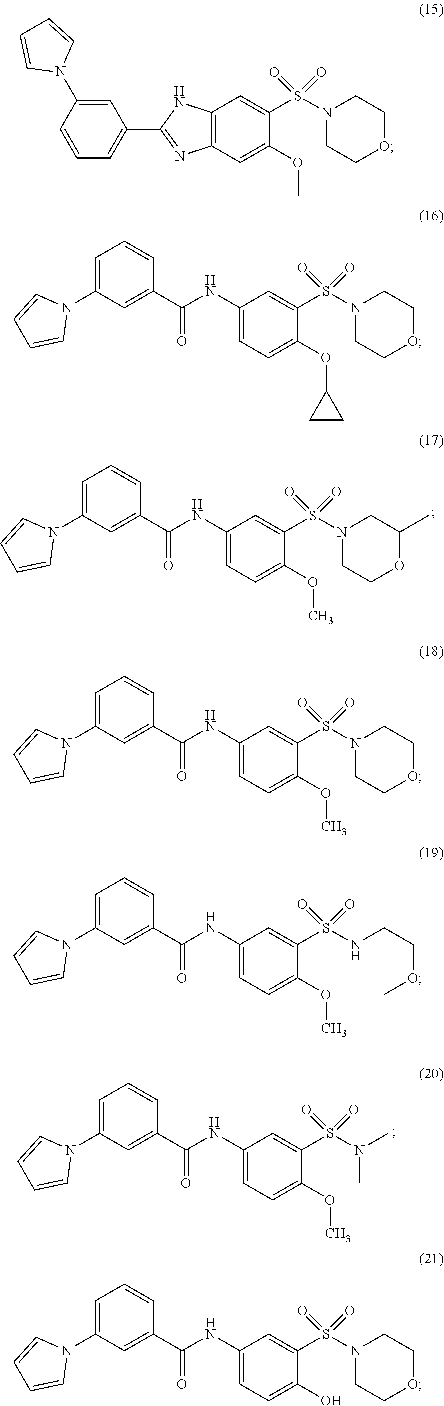

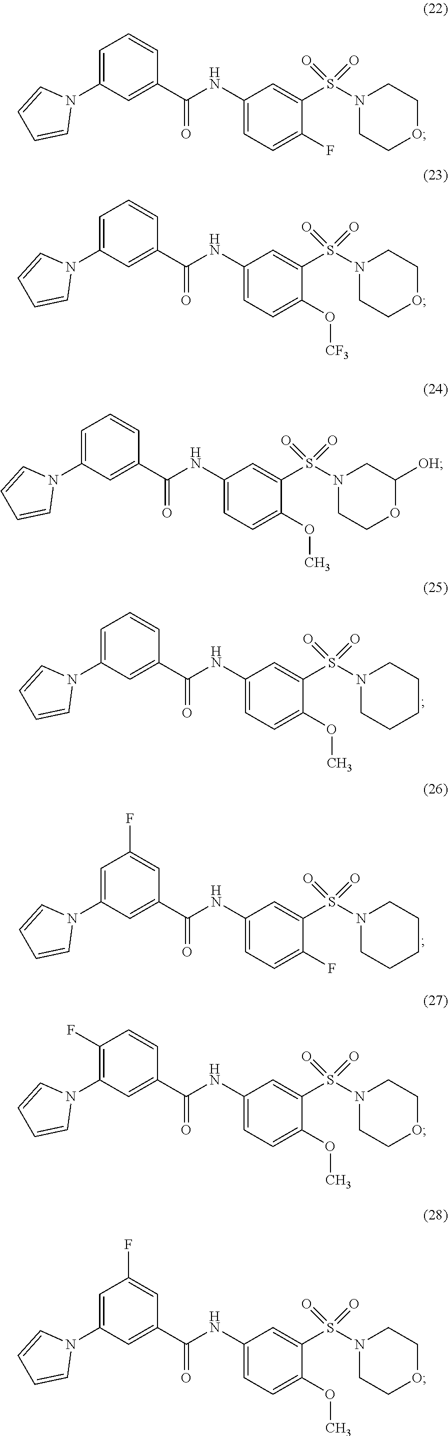

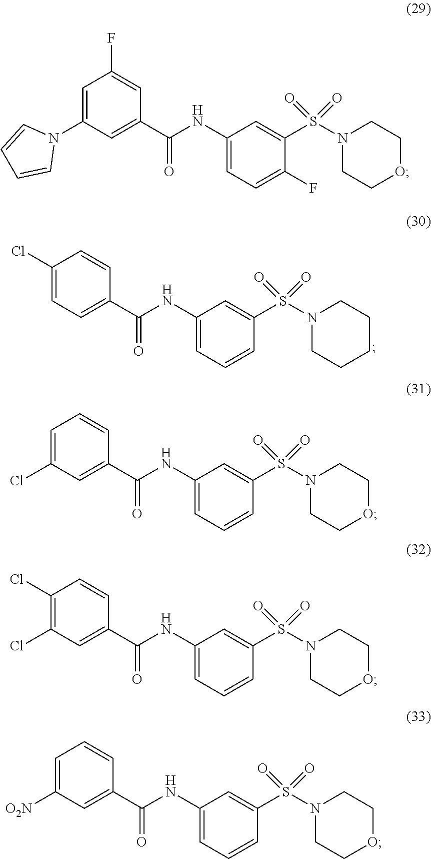 [SMILES: O=C(Nc1ccc(F)c(S(=O)(=O)N2CCOCC2)c1)c1cc(F)cc(-n2cccc2)c1 ; O=C(Nc1cccc(S(=O)(=O)N2CCCCC2)c1)c1ccc(Cl)cc1 ; O=C(Nc1cccc(S(=O)(=O)N2CCOCC2)c1)c1cccc(Cl)c1 ; O=C(Nc1cccc(S(=O)(=O)N2CCOCC2)c1)c1ccc(Cl)c(Cl)c1 ; O=C(Nc1cccc(S(=O)(=O)N2CCOCC2)c1)c1cccc([N+](=O)[O-])c1]

or a pharmaceutically acceptable salt of any of the foregoing.

### Biological Activity of EIF3e Inhibitors

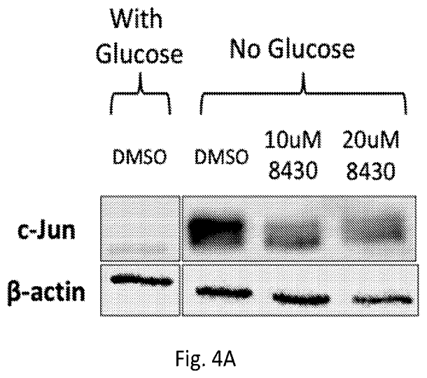

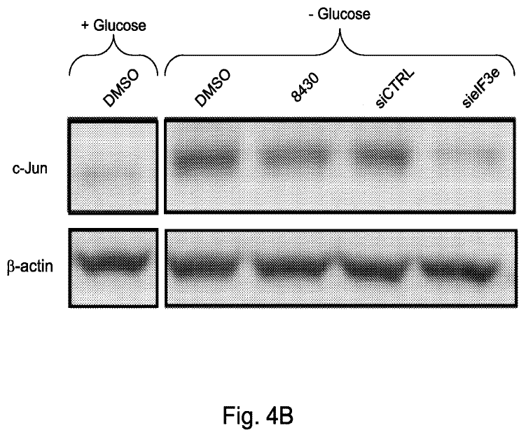

The disclosure provides first in class small molecules that targets the eukaryotic translation initiation factor 3e (eIF3e), a factor known to be critical for the translation of a subset of mRNAs that regulate key processes such as response to stress, epithelial to mesenchymal transition, proliferation and survival, and that is known to be dysregulated in numerous cancers, including breast cancer. Data discussed in this disclosure show that eIF3e regulates cellular stress response pathways by regulating levels of the transcription factor, c-Jun (glucose deprivation stress), the transcription factor HIF-1 (hypoxia stress), and oxidative phosphorylation. eIF3e also regulates cellular levels of both SIX1 and EYA2, a transcription factor and its coactivator that act in a complex to enhance breast cancer metastasis. The inventors identified a small molecule, 8430, by screening a library of molecules that reduce SIX1/EYA2 activity followed by subsequent medicinal chemistry to increase activity. 8430 reduces SIX1/EYA2 protein levels and decreases the SIX1/EYA2 interaction in breast cancer cells. It reverses SIX1-induced transcriptional and metabolic alterations, as well as inhibits TGFβ signaling and EMT in breast cancer cells. Most importantly, 8430 significantly inhibits metastasis of breast cancer cells in mouse models, and this effect is sustained long after administration of 8430 is discontinued. Isothermal Shift Analyses (ITSA) shows that 8430 binds to eIF3e, not directly to Six1 or Eya2. Inventors have discovered that 8430 binds to eIF3e and reduces SIX1/EYA2 protein levels, preventing SIX1/EYA-mediated metastasis in an animal model of metastatic breast cancer. Inventors further discovered that 8430 inhibits stress responses that are thought to facilitate the growth of metastases in new microenvironments as they spread. For example, 8430 inhibits the increase in c-Jun levels in response to glucose deprivation, disrupts translation of the transcription factor HIF-1 (which regulates response to hypoxia), and impacts oxidative phosphorylation (see FIG. 4).

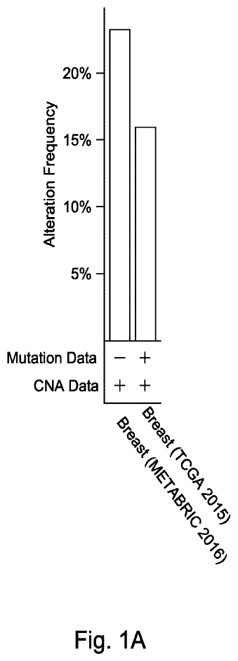

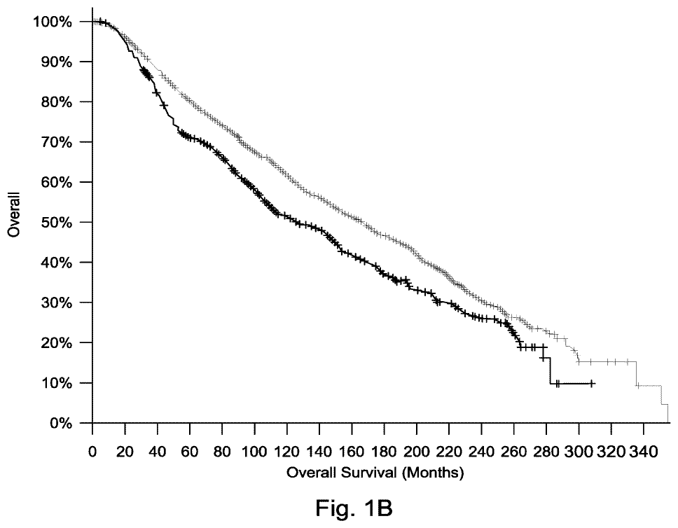

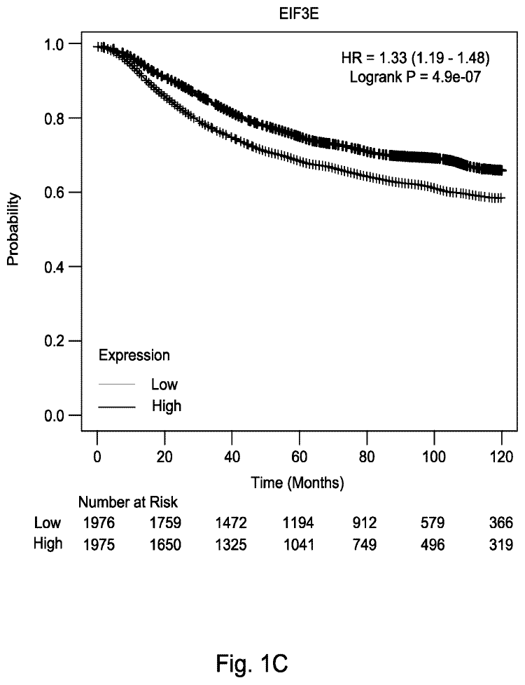

eIF3e in cancer. eIF3e is one of 13 subunits of the eIF3 complex involved in translation initiation in mammalian cells3. eIF3 facilitates the formation and stabilization of the 43S and 48S preinitiation complexes (PIC). Different combinations of subunits non-essential for global translation can form distinct eIF3 subcomplexes which can alter translation efficiency and mRNA specificity. eIF3e is one such subunit whose downregulation does not significantly regulate global mRNA translation, but rather regulates a subset of mRNAs that encode proteins linked to cellular processes such as proliferation, apoptosis, migration/invasion, respiration, response to cellular stress, and differentiation. eIF3e is essential in embryogenesis. The capacity for eIF3e to initiate the translation of tumorigenic and/or metastatic mRNAs suggests a potential oncogenic role for eIF3e, although its role in cancer is complex. eIF3e (also called Int6) is a frequent site of integration of mouse mammary tumor virus (MMTV); MMTV integrates in intronic regions of eIF3e, generating a chimeric transcript which results in mammary tumors in mice. Multiple studies show increased eIF3e mRNA expression in human breast and colon cancers positively correlating with tumor stage. eIF3e is amplified in 23% of oral cancer samples and high eIF3e expression positively correlates with distant metastasis, lymph node metastasis, and worse overall survival in colon cancer. Additionally, the METABRIC and TCGA datasets show that eIF3e is amplified in up to 24% of breast cancer; and eIF3e amplification correlates with worse overall survival (FIG. 1A, B). Because eIF3e is often co-amplified with Myc, we repeated the analysis removing all tumor samples that have co-amplification, and examined only in those tumors that had eIF3e amplification without c-Myc amplification. Even with very low numbers (51) of such tumors, we found a trend towards worsened survival in breast cancer patients that had eIF3e amplified (without c-Myc) vs those that did not (p=0.067; not shown), strongly suggesting that eIF3e promotes worsened survival.

Meta-analysis using Kaplan-Meier Plotter also shows a significant correlation between high eIF3e expression and worse relapse-free survival (FIG. 1C). Furthermore, loss of eIF3e expression results in a reduced migratory/invasive capacity in breast cancer and osteosarcoma models. Similar results were obtained in glioblastoma cell lines in which loss of eIF3e expression resulted in decreased migration and proliferation, and induction of apoptosis.

We have identified a class of eIF3e inhibitors by screening compounds inhibiting the SIX1/EYA2 protein-protein interaction. SIX1 (a member of the SIX family of homeobox genes) is expressed during early embryogenesis. Most adult tissues no longer express SIX1, but many cancers aberrantly re-express SIX1. For example, SIX1 is overexpressed in 50% of primary breast tumors and 90% of metastatic lesions. SIX1 expression in breast cancers is associated with adverse outcomes in patients, including decreased time to metastasis and relapse, and shortened survival.

A preponderance of data in animal models confirms SIX1's role in breast, and other, tumorigenesis, metastasis and drug resistance. (1) Overexpression of SIX1 transforms immortalized (otherwise normal) mammary epithelial cells, forming highly aggressive tumors when injected orthotopically into the mammary glands of nude mice. (2) Mammary specific SIX1 over-expression in a transgenic mouse model leads to the development of neoplastic breast lesions resembling human breast carcinoma. (3) SIX1 overexpression in tumorigenic but normally nonmetastatic MCF7 mammary carcinoma cells leads to both lymphatic and rare bone metastases when orthotopically injected into mice. (4) SIX1 expression promotes peritumoral and intratumoral lymphangiogenesis, lymphatic invasion, and distant metastasis of human breast cancer cells in mouse models. (5) SIX1 overexpression in luminal MCF7 cells significantly increases tumor initiating capability. (6) SIX1 levels are enriched in CD24low/CD44+ breast cancer stem cells xenografted in mice. (7) SIX1 induces metastasis by activating the TGFβ signaling pathway. (8) Knockdown of SIX1 decreases cancer cell proliferation and significantly reduces tumor size and metastasis. (9) SIX1 expression mediates paclitaxel resistance in breast cancer cells.

SIX1 is a transcription factor with no intrinsic activation or repression domains, it requires the EYA cofactors to mediate its transcriptional effects. Four Eya family members exist in mammals (EYA1-4), each containing a divergent N-terminus, an internal Pro-Ser-Thr (PST) rich activation domain, and a highly conserved C-terminal Eya domain (ED) that is responsible for interactions with the SIX family of proteins. EYA co-activators play important roles in SIX1-mediated transcriptional activation, both in normal development and in various diseases. Like SIX1, most EYA family members are expressed in developing tissues, but not in most normal adult tissues. EYA proteins have also been linked to many cancers (such as Wilms' tumor, glioblastoma and ovarian, lung, cervical and breast cancer) in which SIX1 is overexpressed. Furthermore, we demonstrated that knockdown (KD) of EYA2, the most prevalent EYA in SIX1-overexpressing MCF7 cells, inhibits the ability of SIX1 to induce TGFβ signaling, epithelial mesenchymal transition (EMT), and tumor initiating cell (TIC) characteristics, properties that are all associated with SIX1-induced tumorigenesis and metastasis, and that a point mutant of SIX1 that cannot bind EYA2 cannot induce TGFβ signaling, EMT, or metastasis. These data demonstrate a clear requirement for the SIX1/EYA2 interaction in metastasis of breast cancer.

By examining the TCGA breast cancer dataset, we demonstrated that over-expression of SIX1 and EYA2 together, but not either one alone, is significantly associated with shortened time to relapse and metastasis and shortened survival, in both luminal breast cancers (hormone receptor positive, HR+) as well as in basal breast cancers (largely triple negative breast cancers, TNBC). These clinical data strongly indicate EYA proteins are required for SIX1-mediated breast tumor progression.

We have discovered that certain compounds of Formula I reduce SIX1/EYA2 protein levels and decrease the SIX1/EYA2 interaction in breast cancer cells. Certain compounds of Formula I reverse SIX1-induced transcriptional and metabolic alterations, as well as inhibit TGFβ signaling and EMT in breast cancer cells. Most importantly, compound 8430 has been shown to inhibit metastasis of breast cancer cells in mouse models, and this effect is sustained long after administration of the compounds is discontinued. Isothermal Shift Analyses (ITSA) shows eIF3e is a direct cellular target of certain compounds of Formula I.

To confirm whether the inhibition of eIF3e can affect SIX1 and EYA2, thus explaining our observations that SIX1/EYA2 are decreased with treatment with a compound or salt of Formula I, we transiently knocked down expression of eIF3e in the MCF7-SIX1 HR+ breast cancer cells. We observed decreased SIX1 and EYA2 protein expression and a growth defect. Certain compounds of Formula I stabilize purified eIF3e and resulting in a slight decrease in global protein synthesis (which is consistent with multiple studies showing a moderate decrease in global translation initiation with the loss of eIF3e). Furthermore, we have found that 8430 treatment inhibits a glucose deprivation mediated stress response, a known eIF3 response. Our data indicate that compounds of Formula I can potently reduce breast cancer metastasis, likely in part by reducing protein levels of SIX1 and EYA2. We have further shown that the binding of eIF3e by compounds of Formula I affects the translation of other mRNAs and biological processes that together with SIX1 and EYA2 impinge on metastasis, particularly those involved in stress response. FIG. 4 shows that a compound of Formula I, 8430, inhibits levels of c-Jun in response to glucose deprivation.

During the process of metastasis, tumor cells must navigate numerous different stressors and microenvironments, and thus must have mechanisms in place to rapidly respond to different signals. It is becoming increasingly appreciated that mRNA translation is critical for the metastatic process, to enable cells to rapidly adapt to the changing environments they encounter during the metastatic cascade. The disclosure provides first-in-class small molecule inhibitors of eIF3e that specifically affects a subset of mRNA that are critical for the metastatic process. These mRNAs include but are not limited to, stress response genes c-Jun and H1F-1 and SIX1 and EYA2, which are developmental genes that are shut down in normal adult tissue but abnormally re-expressed across different subtypes of breast cancers and drive the metastatic process. Certain compounds of Formula I can also inhibit the growth of a HR+ breast cancer PDX line that harbors a mutation in the estrogen receptor that makes it resistant to endocrine therapy, providing an alternate therapy to prevent metastasis of endocrine therapy resistant tumors. It should also be noted that in an immune competent model (BALB/c) of triple negative breast cancer (TNBC), using the 66c14 cells that we have previously demonstrated are dependent on SIX1 to metastasize. Similarly, 3 weeks of 8430 treatment trended towards a reduction in metastasis (not shown; low numbers of animals were used with high variability, p-value=0.08).

### Methods of Treatment

The compounds/pharmaceutical compositions/combinations disclosed herein are useful for treating disease and disorders in patients in which eIF3e inhibition, reducing or altering the interaction of eIF3e and eIF3d, or eIF3e and any other proteins with which it interacts, reducing c-Jun or HIF1a levels, or inhibiting SIX1/EYA2 interactions is beneficial. For example, compounds of Formula I are useful for treating cancer, such as metastatic breast cancer (of any subtype), glioblastoma, Wilms' tumor, ovarian cancer, lung cancer, cervical, oral cancer, lymphoma, and osteosarcoma. Compounds of Formula I may be used to treat breast cancers that have become insensitive to hormone therapies, such as ER⁺ tumors. Compounds of the disclosure are also useful for delaying or reducing cancer cell resistance to chemotherapeutic agents including paclitaxel.

An effective amount of a pharmaceutical composition of the disclosure includes an amount sufficient to (a) inhibit the progression of cancer; (b) cause a remission; or (c) cause a cure of cancer, or (d) significantly reduce the level of cancer markers in a patient's blood, serum, or tissues.

This disclosure provides a method of reducing primary tumor growth, reducing primary tumor size, as well as reducing tumor metastasis. In some cases metastasis and tumor volume may be reduced to the point of being undetectable. The disclosure includes a method of treating cancer in a patient having a primary breast tumor, or metastatic breast cancer, including cancers which are insensitive to hormone therapies, such as ER⁺ tumors that are hormone resistant and TNBC-tumors, comprising administering an effective amount of a compound of Formula I to produce a remission.

An effective amount of a pharmaceutical composition described herein will also provide a sufficient concentration of the active agents in the concentration when administered to a patient. A sufficient concentration of an active agent is a concentration of the agent in the patient's body necessary to reduce cancer symptoms or slow cancer progression. Such an amount may be ascertained experimentally, for example by assaying blood concentration of the agent, or theoretically, by calculating bioavailability.

According to the methods of the disclosure, the compound or pharmaceutically acceptable salt of Formula I and at least one additional active agent may be: (1) co-formulated and administered or delivered simultaneously in a combined formulation; (2) delivered by alternation or in parallel as separate formulations; or (3) by any other combination therapy regimen known in the art. When delivered in alternation therapy, the methods of the disclosure may comprise administering or delivering the compound or salt of Formula I and an additional active agent sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

Methods of treatment and pharmaceutical combinations including compounds or pharmaceutically acceptable salts of Formula I described herein together with any one or combination of the following compounds and substances as an additional active agent are provided by the disclosure:

Methods of inhibiting eIF3e in vivo comprise providing a compound or pharmaceutically acceptable salt of Formula I to a patient having cancer, a concentration of the compound or salt of Formula I sufficient to inhibit eIF3e in vitro are included herein. In this instance the concentration includes an in vivo concentration, such as a blood or plasma concentration. The concentration of compound sufficient to inhibit eIF3e in vitro may be determined from an assay of eIF3e inhibition such as the assay provided in Example 5.

Methods of treatment include providing certain dosage amounts of a compound or pharmaceutically acceptable salt of Formula I to a patient. Dosage levels of each active agent of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single unit dosage form will vary depending upon the patient treated and the particular mode of administration. In certain embodiments about 0.1 mg to about 2000 mg, from about 10 mg to about 1500 mg, from about 100 mg to about 1000 mg, from about 200 mg to about 800 mg, or from about 300 to about 600 mg of a compound of Formula I and optionally from about 0.1 mg to about 2000 mg, from about 10 mg to about 1500 mg, from about 100 mg to about 1000 mg, from about 200 mg to about 800 mg, or from about 300 to about 600 mg of a compound of an additional active agent, for example paclitaxel, or other chemotherapeutic agent are provided to a patient. It is preferred that each unit dosage form contains less than 1200 mg of active agent in total. Frequency of dosage may also vary depending on the compound used and the particular disease treated. Length of dosage may also vary depending on the compound used and the particular disease treated.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease in the patient undergoing therapy.

### EXAMPLES

### Abbreviations

DCM Dichloromethane
EtOAc Ethyl Acetate
HATU Hexafluorophosphate Azabenzotriazole Tetramethyl Uronium

### Example 1. Synthesis of Compound 8430

The synthesis of 8430 was initiated by the nucleophilic substitution of 2-methoxy-5-nitrobenzenesulfonyl chloride with morpholine followed by a palladium-on-carbon catalyzed hydrogenation of the nitro group to the aniline. This aniline was then subjected to HATU-mediated amide coupling with commercially available 3-(1H-pyrrol-1-yl)benzoic acid to provide 8430 (N-(4-methoxy-3-(morpholinosulfonyl)phenyl)-3-(1H-pyrrol-1-yl)benzamide).

Step 1. 4-Methoxy-3-(morpholinosulfonyl) aniline: Morpholine (0.519 ml, 5.96 mmol) was added dropwise to a solution of 2-methoxy-5-nitrobenzenesulfonyl chloride (1.00 g, 3.97 mmol) in DCM (15 mL) at RT. Warming (bubbling) was observed during addition of morpholine. Triethylamine (0.831 mL, 5.96 mmol) was added (some precipitation was observed). The mixture was stirred at RT for 16 hrs. and then treated with water. The mixture was extracted with more DCM, the organic layer was washed with saturated aqueous HaHCO₃, dried (MgSO₄), filtered, concentrated and analyzed by LCMS and ¹H NMR. ¹H NMR indicated that morpholine was present in the mixture. However, this material 4-((2-methoxy-5-nitrophenyl)sulfonyl)morpholine was not purified further but dissolved in ethanol (50 ml) and DCM (25 ml). Under a stream on nitrogen 10% Pd/C (100 mg) was added and the reaction was stirred under a balloon pressure of hydrogen. It was analyzed after 5 hr. Starting material was still left but the required product aniline was observed by LCMS. Another 50 mg of 10% Pd/C (thus, total 10% Pd—C added was 150 mg, 0.141 mmol) and left stirring under hydrogen balloon pressure for approximately 16 hr. The mixture was filtered under a stream of nitrogen through a pad of Celite®, concentrated, and purified via flash silica gel chromatography with a 0 to 50% EtOAc/DCM gradient to provide 4-methoxy-3-(morpholinosulfonyl)aniline (0.96 g, 3.53 mmol, 89% yield). 1H NMR (400 MHz, DMSO-d6) δ 7.02-6.96 (m, 2H), 6.81 (dd, J=8.8, 2.9 Hz, 1H), 5.08 (s, 2H), 3.73 (s, 3H), 3.63-3.55 (m, 4H), 3.06-2.98 (m, 4H).

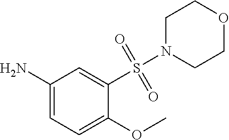 [SMILES: COc1ccc(N)cc1S(=O)(=O)N1CCOCC1]

Synthesis of N-(4-methoxy-3-(morpholinosulfonyl)phenyl)-3-(1H-pyrrol-1-yl)benzamide (8430). 4-Methoxy-3-(morpholinosulfonyl)aniline (0.58 g, 2.13 mmol) was taken in DMF (7.10 ml), treated with 3-(1H-pyrrol-1-yl)benzoic acid (0.498 g, 2.66 mmol), HATU (1.215 g, 3.19 mmol), and diisopropylethylamine (0.56 ml, 3.19 mmol) in a round bottom flask that was flooded with N₂ and a septum fixed on top. The reaction was stirred at RT for approximately 16 hr. The reaction was diluted with EtOAc and washed with saturate aqueous NH₄Cl. The EtOAc layer was dried (MgSO₄), filtered and dried under vacuum. The residue was dissolved in minimal DCM and purified via silica gel flash chromatography with a 0 to 100% gradient of EtAOc in hexanes. Fractions were analyzed by TLC and concentrated and analyzed by ¹H NMR that revealed that compound had residual DMF. The compound was taken up in minimal DCM to make a suspension, sonicated and then filtered through a fritted plastic funnel, under house vacuum. The residue was air dried overnight and then analyzed by ¹H NMR to reveal that the DMF has been removed. Yield: N-(4-methoxy-3-(morpholinosulfonyl)phenyl)-3-(1H-pyrrol-1-yl)benzamide (699 mg, 1.583 mmol, 74.3% yield). ¹H NMR (400 MHz, DMSO-d6) δ 10.45 (s, 1H), 8.22 (d, J=2.7 Hz, 1H), 8.11 (m, 1H), 8.08 (dd, J=8.9, 2.7 Hz, 1H), 7.85-7.77 (m, 2H), 7.61 (t, J=7.9 Hz, 1H), 7.48 (t, J=2.2 Hz, 2H), 7.31 (d, J=9.1 Hz, 1H), 6.32 (t, J=2.2 Hz, 2H), 3.90 (s, 3H), 3.61 (app t, J=4.7 Hz, 4H), 3.10 (app t, J=4.7 Hz, 4H).

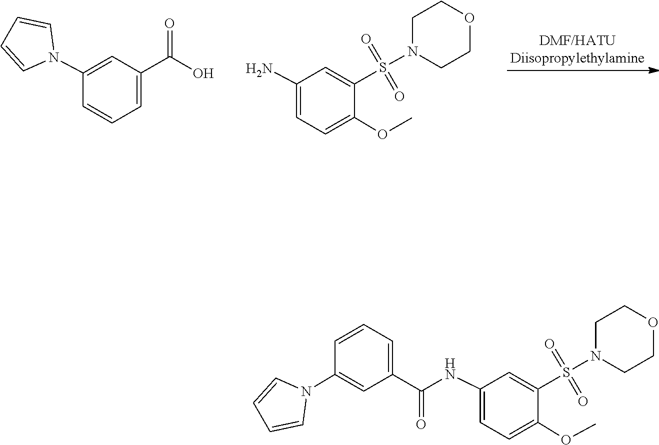 [SMILES: O=C(O)c1cccc(-n2cccc2)c1 ; COc1ccc(N)cc1S(=O)(=O)N1CCOCC1 ; COc1ccc(NC(=O)c2cccc(-n3cccc3)c2)cc1S(=O)(=O)N1CCOCC1]

Other compounds of Formula I can be synthesized according to this procedure

### Example 2. 8430 Stabilizes eIF3e in Isothermal Shift Analysis

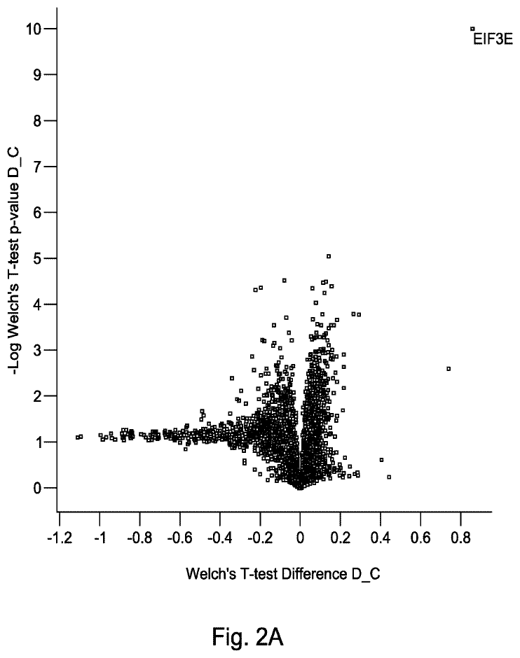

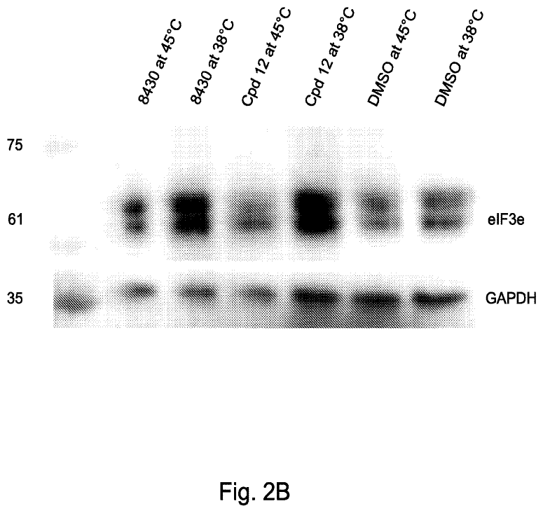

ITSA demonstrated that eIF3e was significantly stabilized (remaining in the supernatant) by 8430, compared to all other proteins in the cell (FIG. 2A). In addition, a chemically related but inactive analog did not increase the stability of eIF3e, demonstrating specificity of 8430 (data not shown). We confirmed the stabilization effect of 8430 on purified eIF3e using Western blot analysis of proteins that remained in the soluble fraction at an elevated temperature (FIG. 2B). These data strongly suggest that 8430 directly targets eIF3e, which then affects the translation and/or degradation of stress response proteins and SIX1 and EYA2, leading to the cellular and in vivo phenotypes we observed.

### Example 3. 8430 has Effect on Global Translation in Breast Cancer Cells

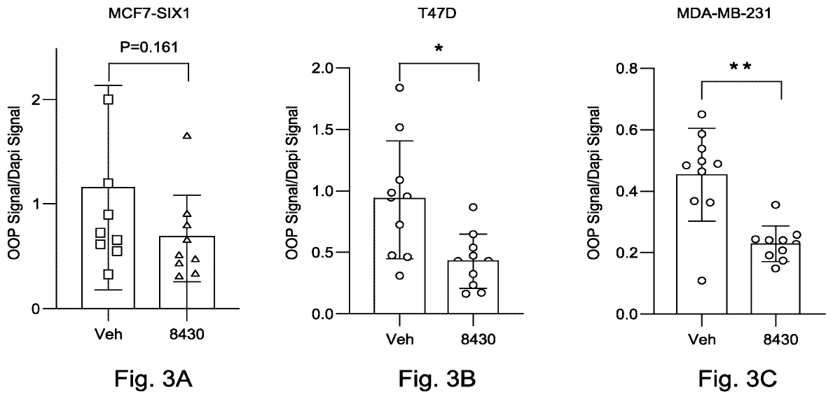

If 8430 is inhibiting eIF3e, it will partially but not completely inhibit global translation, given that eIF3e is known to influence the translation subsets of mRNAs. In support of this idea, 8430 treatment of all three models used above demonstrates a reduction in translation, though significance was only reached in the T47D and MDA-MB-231 systems (FIG. 3).

### Example 4. 8430 Reverses the Increase of c-Jun Protein Levels Induced by Glucose Starvation

Of interest, eIF3d, a binding partner of eIF3e, has recently been implicated in the response to metabolic stress (specifically glucose deprivation) via its ability to increase translation of c-Jun. Because eIF3d and e are physically associated in the eIF3 complex, we determined whether both eIF3e knockdown and 8430 could inhibit c-Jun upregulation in response to glucose deprivation. As seen in FIG. 4, both 8430 treatment and eIF3e KD reduce the increase in c-Jun protein levels observed in response to glucose deprivation. Taken together, these data support that eIF3e is a target of 8430.

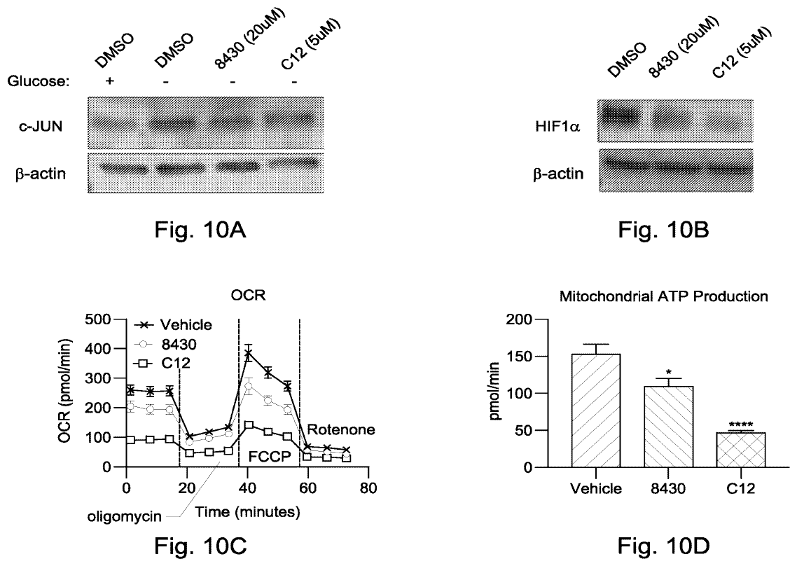

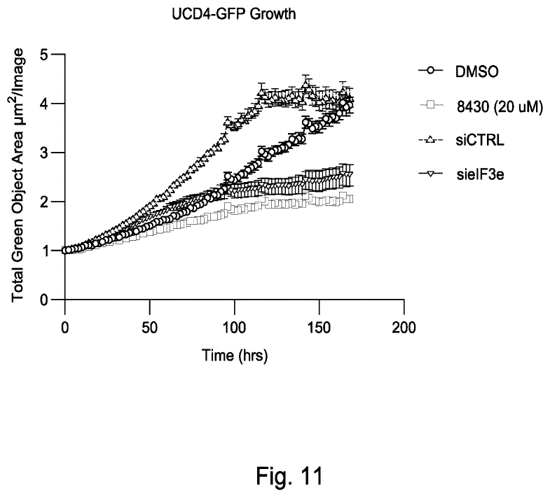

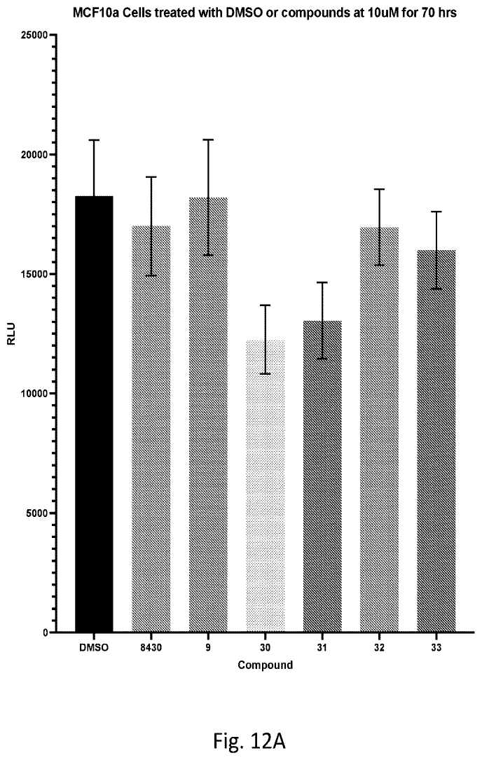

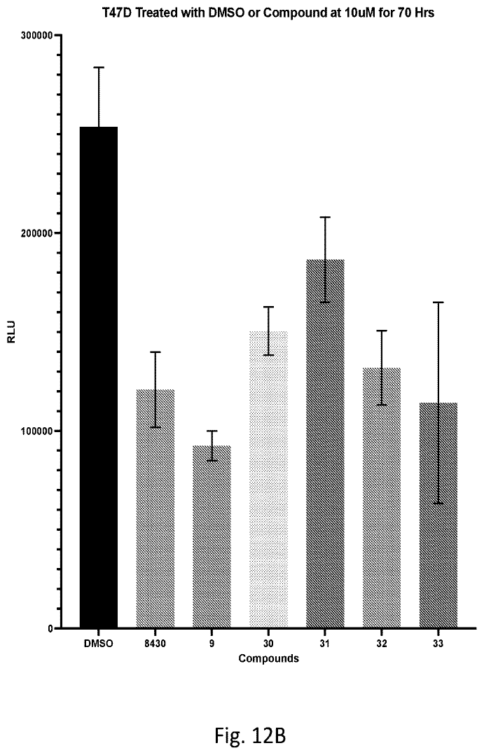

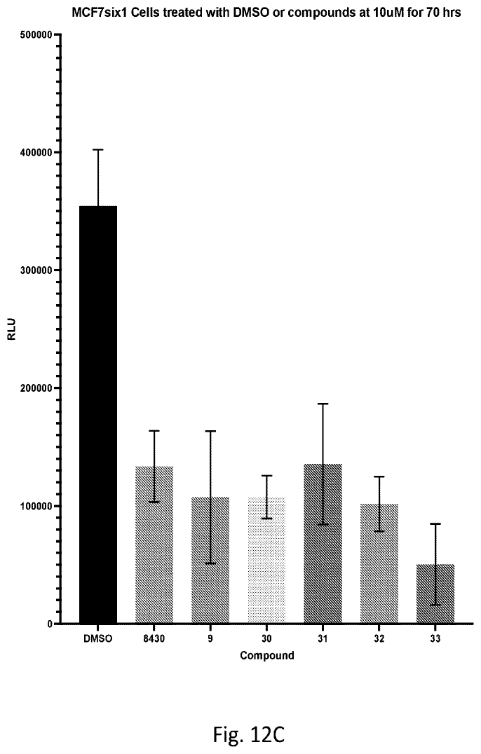

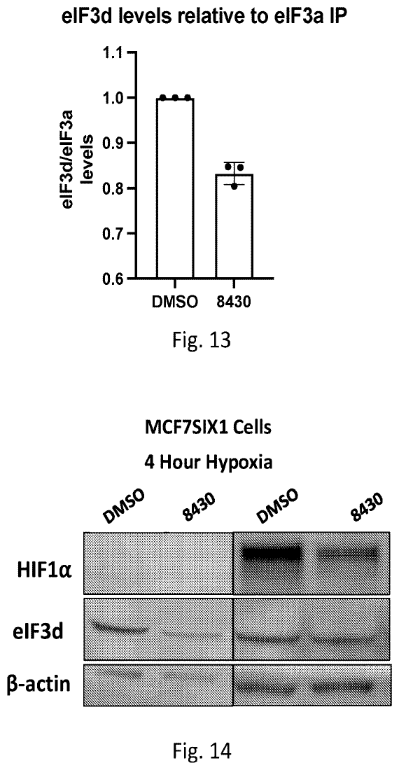

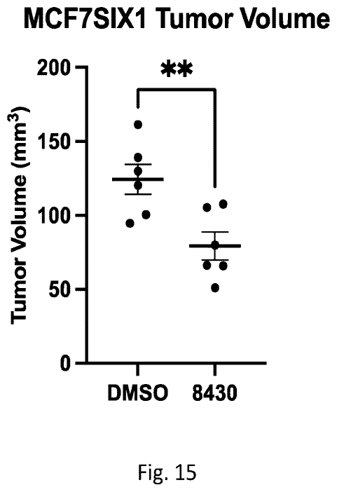

Additionally, loss of eIF3e has previously been shown to prevent the translation of HIF1α in response to hypoxia. 8430 treatment significantly reduces HIF1α protein in response to hypoxia in MCF7-SIX1 cells (FIG. 10) and HEK293T cells (not shown). Lastly, eIF3e is important for mitochondrial homeostasis and the translation of electron transport chain proteins necessary for mitochondrial-dependent ATP production^{10,42}. Preliminary Seahorse Assays on HEK293T cells show a decrease in oxygen consumption rate and mitochondrial ATP production with 8430 treatment.

### Example 5. Transient Knock-Down of eIF3e in MCF7-SiX1 Cells Mimics 8430 Treatment

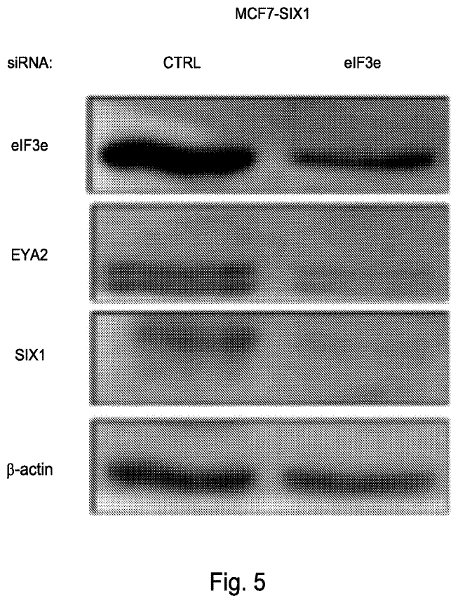

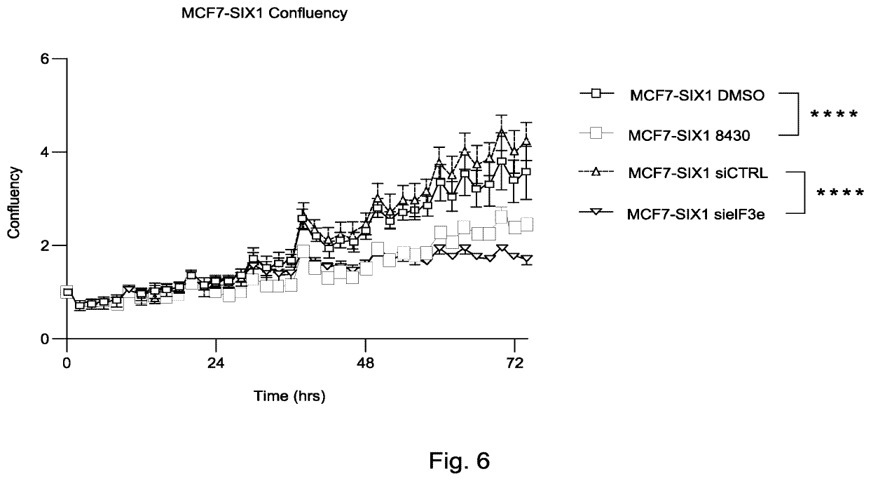

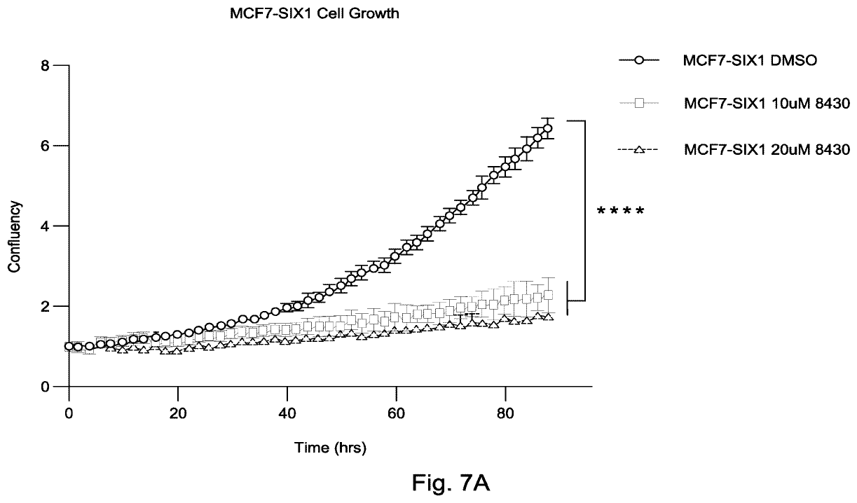

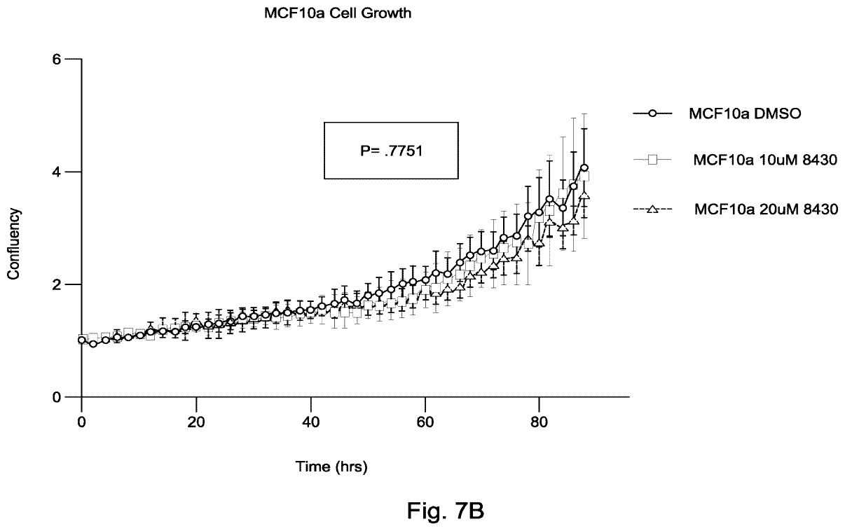

As seen with 8430 treatment, transient KD of eIF3e also reduces SIX1 and EYA2 levels (FIG. 5). Importantly, loss of eIF3e phenocopies the in vitro growth defect observed with 8430 treatment (FIG. 6). It should be noted that this level of growth defect is not seen in the MCF7-Ctrl cells treated with 8430 (not shown), demonstrating that 8430 is specifically targeting phenotypes observed in SIX1-overexpressing cells. Further, we observed no effect of 8430 treatment in the immortalized, but non-tumorigenic MCF10A cell line, suggesting that our eIF3e inhibitors have specific effects in cancer cells (FIG. 7).

### Example 6. 8430 Analogs Demonstrate Similar or Better Potency

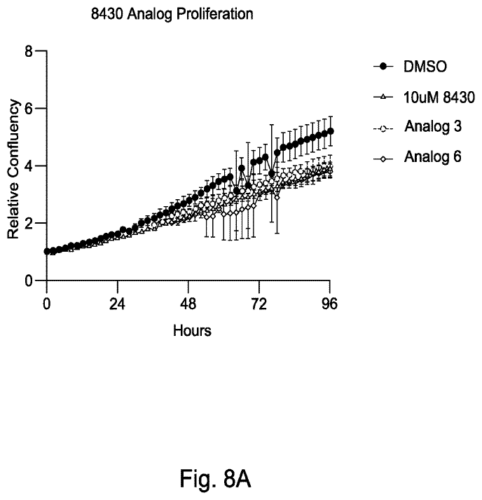

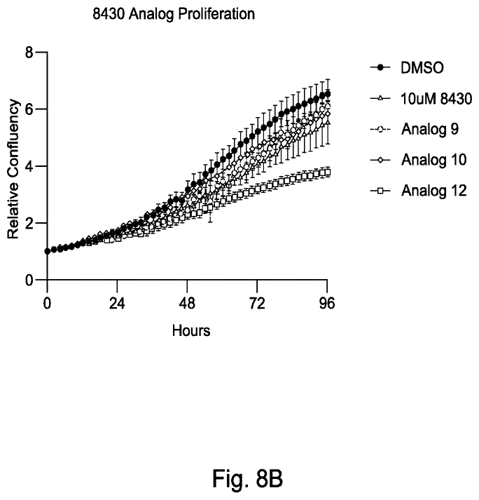

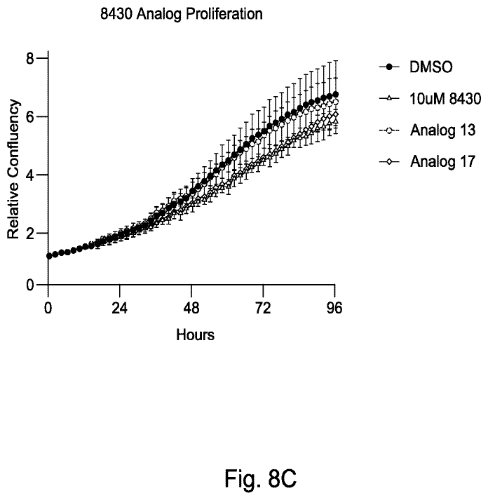

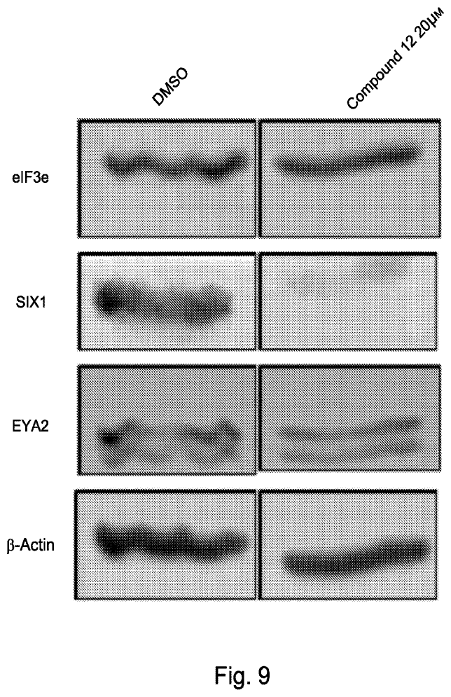

Although 8430 has no severe toxicity at even the highest dose tested (25 mg/kg), it has a very short in vivo half-life in mice (0.25 hrs.). Due to this limitation, we directly injected 8430 into the tumor site in our proof-of-concept animal experiments. To improve the pharmacological properties of 8430, we synthesized a number of analogs (See exemplified compounds of Formula I in the DETAILED DESCRIPTION section). We evaluated the effect of these compounds on growth of MCF7-SIX1 cells using Incucyte live cell imaging as the most rapid and efficient way to test the potency of analogs. We found that compound 12 inhibits the proliferation of these cells much more effectively than 8430 (FIG. 8), and that this compound also reduces SIX1 and EYA2 levels (FIG. 9), similar to 8430 and eIF3e KD (FIG. 5). We further confirmed that compound 12 stabilizes purified eIF3e (FIG. 2B). Compounds 3, 6, 9, 10, and 17 also have eIF3e inhibitory activity and may have improved pharmacological properties over 8430 (FIG. 8).

### Example 7. 8430 Inhibits Growth of ER+ PDX Breast Cancer Cell Line (UCD4)

Preliminary results demonstrate that both 8430 and eIF3e KD similarly inhibit the growth of UCD4 PDX line cells (FIG. 10), which are derived from an ER+ breast cancer. This result is particularly exciting, as the UCD4 line is HR+, but harbors a D538G mutation in the ER, thus rendering it insensitive to hormone therapies such as tamoxifen. Compounds of Formula I may work in ER+ tumors as they become resistant to anti-hormone therapies, and ultimately metastasize.

1. A compound of the formula 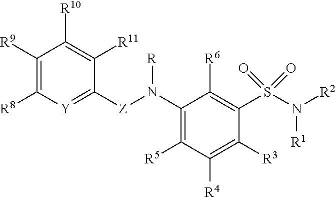
or a pharmaceutically acceptable salt thereof, wherein
Y is N or CR⁷;
Z is methylene (—CH₂—), keto (—(C═O)—), or is taken together with R⁵ to form a 5-membered partially unsaturated ring that contains 1 or 2 nitrogen atoms;
R is hydrogen or methyl;
R¹ is C₁-C₆alkyl or (C₃-C₆cyclopropyl)C₀-C₂alkyl; and
R² is hydrogen or C₁-C₆alkyl; or
R¹ and R² are taken together to form a 5- or 6-membered heterocycloalkyl group optionally containing one additional heteroatom selected from N, O, and S, and optionally substituted with one or two substituents independently chosen from halogen, hydroxyl, C₁-C₄alkyl, C₁-C₄alkoxy, C₁-C₂haloalkyl, and C₁-C₂haloalkoxy;
R³ is halogen, hydroxyl, C₁-C₆alkyl, C₁-C₆alkoxy C₁-C₂haloalkyl, C₁-C₂haloalkoxy, or (C₃-C₆cycloalkyl)C₀-C₂alkyl-O—;
R⁴, R⁶, R⁷, and R¹¹ are independently hydrogen, halogen, hydroxyl, C₁-C₄alkyl, C₁-C₄alkoxy, C₁-C₂haloalkyl, and C₁-C₂haloalkoxy;
R⁵ is hydrogen, halogen, hydroxyl, C₁-C₄alkyl, C₁-C₄alkoxy, C₁-C₂haloalkyl, and C₁-C₂haloalkoxy, or is taken together with Z to form a 5-membered partially unsaturated ring that contains 1 or 2 nitrogen atoms;
one of R⁸ and R⁹ is halogen, nitro, phenyl, di-C₁-C₄alkylamino, or a 4-6 membered saturated, partially unsaturated or aromatic heterocyclic group containing 1 or 2 additional heteroatoms selected from N, O, and S, each of which is optionally substituted with one or more substituents independently chosen from halogen, hydroxyl, cyano, amino, C₁-C₄alkyl, C₁-C₄alkoxy, C₁-C₂haloalkyl, and C₁-C₂haloalkoxy, and the other of R⁸ and R⁹ is hydrogen, halogen, hydroxyl, cyano, amino, C₁-C₂alkyl, C₁-C₂alkoxy, C₁-C₂haloalkyl, and C₁-C₂haloalkoxy;
R¹⁰ is hydrogen, halogen hydroxyl, cyano, amino, C₁-C₆alkyl, C₁-C₆alkoxy, mono- or di-C₁-C₆alkylamino, mono- or di-alkylamide, C₁-C₂haloalkyl, and C₁-C₂haloalkoxy where any alkyl or alkoxy at R¹, R², R³, or R¹⁰ optionally has one methylene of the alkyl chain replaced by O, NH, N(C₁-C₄alkyl), or S and where the following compounds are excluded

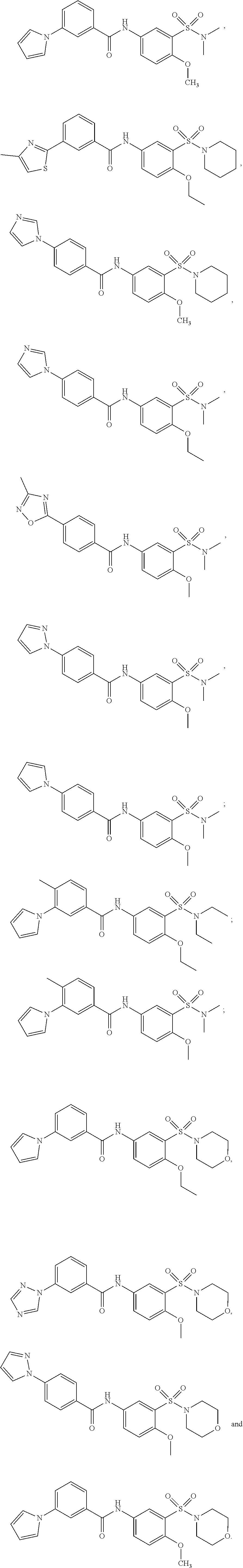

2. The compound or pharmaceutically acceptable salt of claim 1, wherein
R¹ and R² are taken together to form a 6-membered heterocycloalkyl optionally containing one additional heteroatom selected from N, O, and S, and optionally substituted with one or two substituents independently chosen from halogen, hydroxyl, C₁-C₄alkyl, C₁-C₄alkoxy, C₁-C₂haloalkyl, and C₁-C₂haloalkoxy.

3. The compound or pharmaceutically acceptable salt of claim 1, wherein
R¹ and R² are taken together to form a piperazine, piperidine, or morpholine ring that is optionally substituted with one or two methyl substituents: or
R¹ and R² are both methyl or both ethyl.

4. (canceled)

5. The compound or pharmaceutically acceptable salt of claim 1, where
R³ is halogen, hydroxyl, methoxy, ethoxy, or cycloalkyloxy; and R⁴, R⁵, and R⁶ are all hydrogen; and Z is a carbonyl group.

6. The compound or pharmaceutically acceptable salt of claim 1, of the formula:

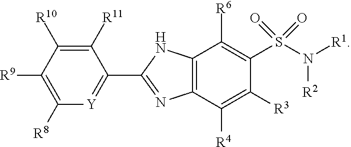

7-8. (canceled)

9. The compound or pharmaceutically acceptable salt of claim 1, Y is CR⁷ and R⁷ is hydrogen or methyl.

10. The compound or pharmaceutically acceptable salt of claim 1, where R⁹, R¹⁰, and R¹¹ are all hydrogen.

11. (canceled)

12. The compound or pharmaceutically acceptable salt of claim 1, where R⁸ is phenyl, di-methylamino, or a 4-, 5-, or 6-membered heterocycloalkyl ring optionally containing one additional heteroatom selected from N, O, and S, and optionally substituted with methyl or R⁸ is a 5-membered heteroaryl ring optionally containing one or two heteroatoms selected from N, O, and S and optionally substituted with halogen, C₁-C₂alkyl, C₁-C₂alkoxy, trifluoromethyl, and trifluoromethoxy; and
R⁹ is hydrogen or methyl.

13. The compound or pharmaceutically acceptable salt of claim 1, where
R⁸ is hydrogen or methyl; and
R⁹ is phenyl, di-methylamino, or a 4-, 5-, or 6-membered heterocycloalkyl ring optionally containing one additional heteroatom selected from N, O, and S. and optionally substituted with methyl or R⁸ is phenyl or a 5-membered heteroaryl ring optionally containing one or two additional heteroatoms selected from N, O, and S and optionally substituted with halogen, C₁-C₂alkyl, C₁-C₂alkoxy, trifluoromethyl, and trifluoromethoxy; and R⁹ is hydrogen or methyl.

14. The compound or pharmaceutically acceptable salt claim 1, where R¹⁰ is hydrogen, halogen, C₁-C₂alkyl, C₁-C₂alkoxy, or dimethylaminoethylcarboxamide.

15. The compound or pharmaceutically acceptable salt of claim 1, where
R¹ and R² are both methyl or ethyl, or
R¹ and R² are taken together to form a morpholine, piperazine, or piperidine ring, each of which is optionally substituted with one or two methyl substituents.
R³ is halogen, hydroxyl, methoxy, or cyclopropyloxy;
R⁴, R⁵, and R⁶ are all hydrogen;
R⁷ and R¹¹ are both hydrogen or methyl;
one R⁸ and R⁹ is hydrogen, halogen, or methyl; and the other of R⁸ and R⁹ is a halogen, nitro, 4-, 5-, or 6-membered heterocycloalkyl ring optionally containing one additional heteroatom selected from N, O, and S, and optionally substituted with methyl or R⁸ is phenyl or a 5-membered heteroaryl ring optionally containing one or two additional heteroatoms selected from N, O, and S each of which phenyl and 5-membered heteroaryl are optionally substituted with halogen, C₁-C₂alkyl, C₁-C₂alkoxy, trifluoromethyl, and trifluoromethoxy; and R⁹ is hydrogen or methyl; and
R¹⁰ is hydrogen, halogen, C₁-C₂alkyl, C₁-C₂alkoxy, or dimethylaminoethylcarboxamide.

16. The compound of claim 1, where the compound is 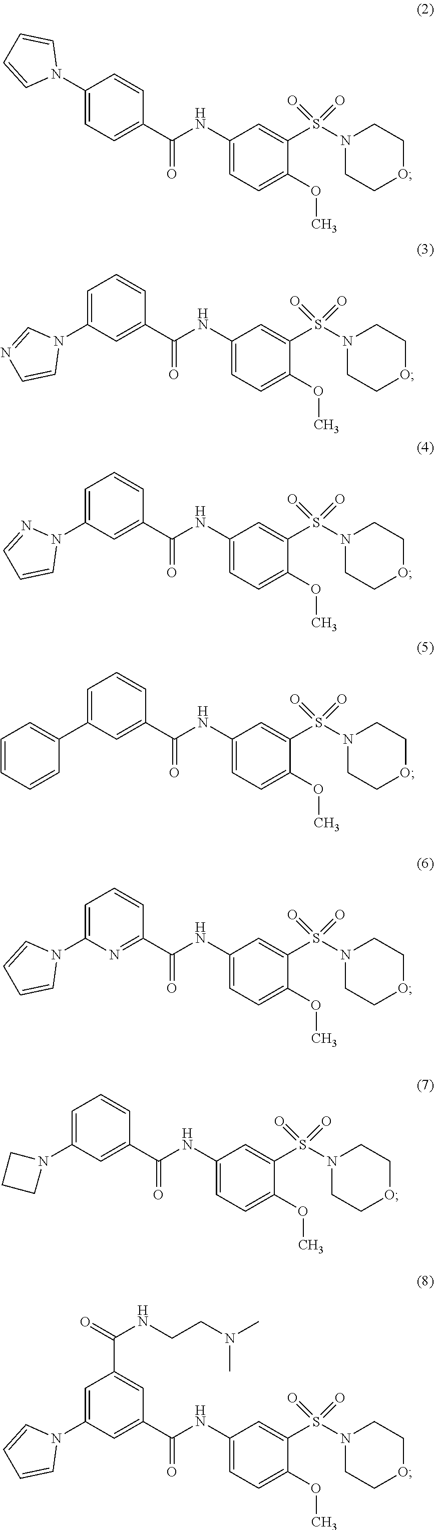 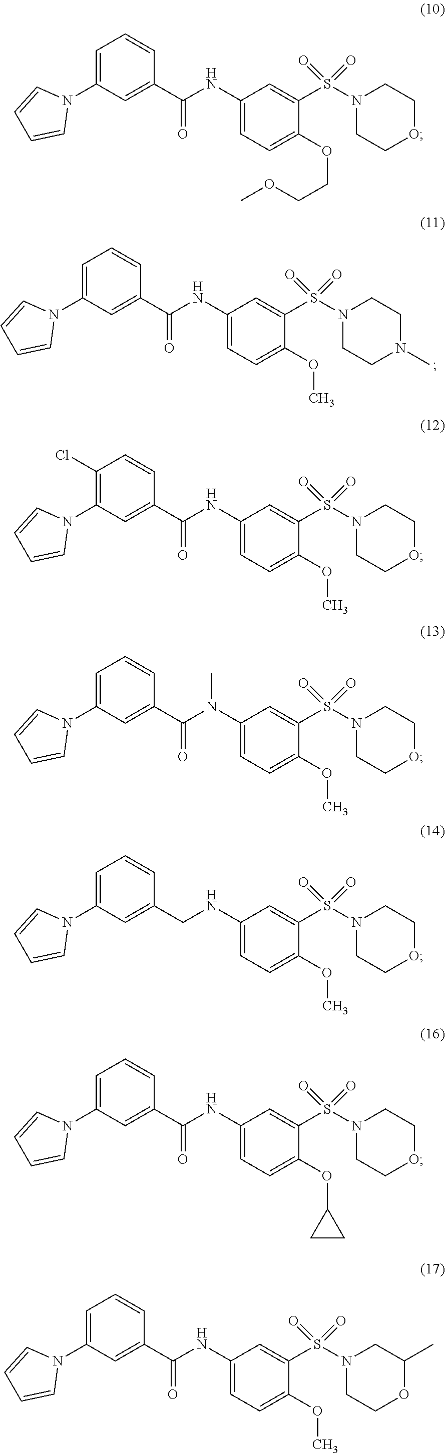 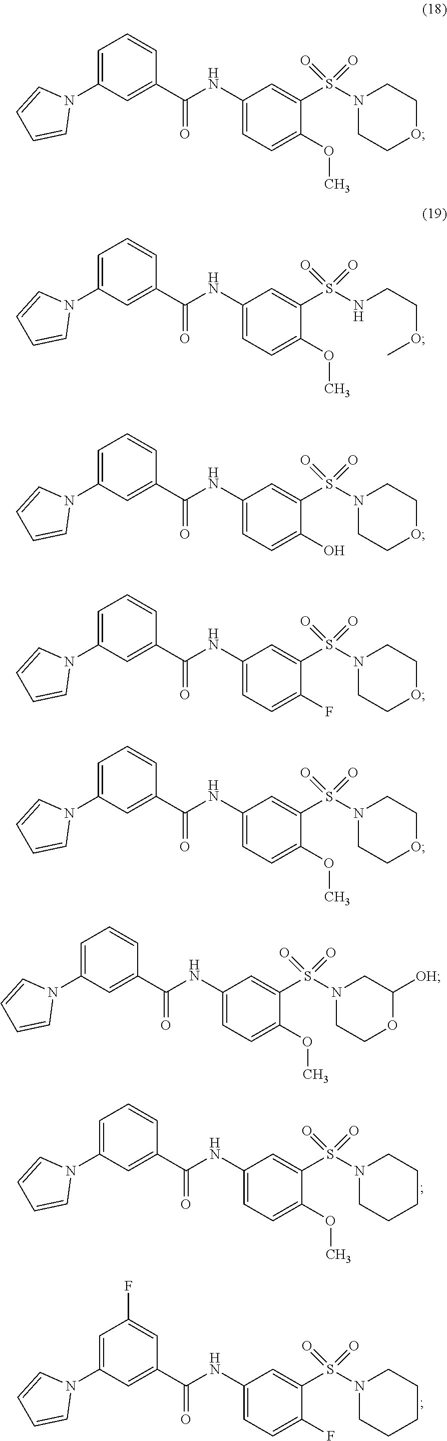 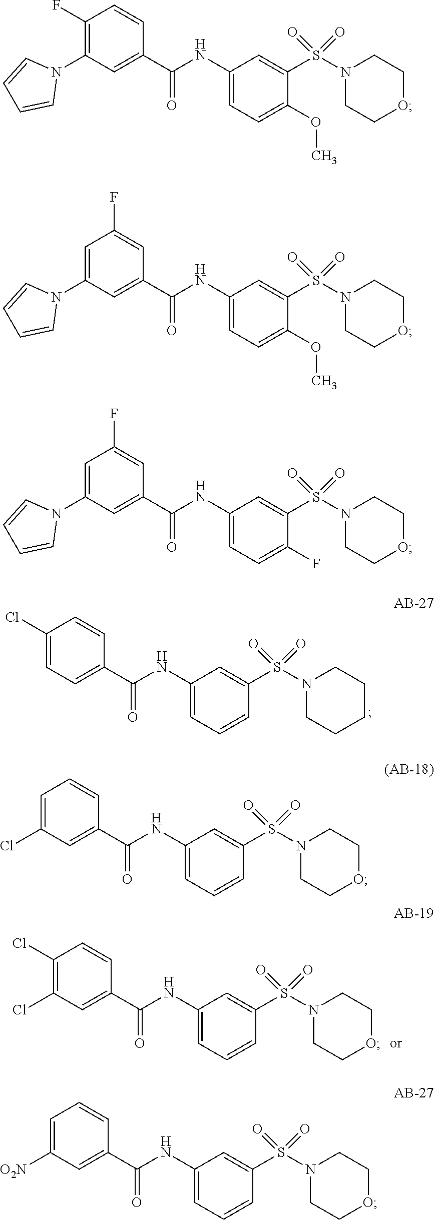
or a pharmaceutically acceptable salt of any of the foregoing.

17. A pharmaceutical composition comprising a compound or pharmaceutically acceptable salt of claim 1, together with at least one pharmaceutically acceptable excipient.

18. A method of inhibiting a eukaryotic translation initiation factor 3e (eIF3e) comprising contacting the eIF3e with a compound of the formula 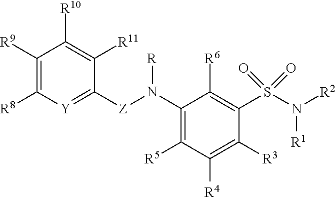
or a pharmaceutically acceptable salt thereof, wherein
Y is N or CR⁷;
Z is methylene (—CH₂—), keto (—(C═O)—), or is taken together with R⁵ to form a 5-membered partially unsaturated ring that contains 1 or 2 nitrogen atoms;
R is hydrogen or methyl;
R¹ is C₁-C₆alkyl or (C₃-C₆cyclopropyl)C₀-C₂alkyl; and
R² is hydrogen or C₁-C₆alkyl; or
R¹ and R² are taken together to form a 5- or 6-membered heterocycloalkyl group optionally containing one additional heteroatom selected from N, O, and S, and optionally substituted with one or two substituents independently chosen from halogen, hydroxyl, C₁-C₄alkyl, C₁-C₄alkoxy, C₁-C₂haloalkyl, and C₁-C₂haloalkoxy;
R³ is halogen, hydroxyl, C₁-C₆alkyl, C₁-C₆alkoxyC₁-C₂haloalkoxy, or (C₃-C₆cycloalkyl)C₀-C₂alkyl-O—;
R⁴, R⁶, R⁷, and R¹¹ are independently hydrogen, halogen, hydroxyl, C₁-C₄alkyl, C₁-C₄alkoxy, C₁-C₂haloalkyl, and C₁-C₂haloalkoxy;
R⁵ is hydrogen, halogen, hydroxyl, C₁-C₄alkyl, C₁-C₄alkoxy, C₁-C₂haloalkyl, and C₁-C₂haloalkoxy or R⁵ is taken together with R⁵ to form a 5-membered partially unsaturated ring that contains 1 or 2 nitrogen atoms;
one of R⁸ and R⁹ is halogen, nitro, phenyl, di-C₁-C₄alkylamino, or a 4-6 membered saturated, partially unsaturated or aromatic heterocyclic group containing 1 or 2 additional heteroatoms selected from N, O, and S, each of which is optionally substituted with one or more substituents independently chosen from halogen, hydroxyl, cyano, amino, C₁-C₄alkyl, C₁-C₄alkoxy, C₁-C₂haloalkyl, and C₁-C₂haloalkoxy, and the other of R⁸ and R⁹ is hydrogen, halogen, hydroxyl, cyano, amino, C₁-C₂alkyl, C₁-C₂alkoxy, C₁-C₂haloalkyl, and C₁-C₂haloalkoxy;
R¹⁰ is hydrogen, halogen hydroxyl, cyano, amino, C₁-C₆alkyl, C₁-C₆alkoxy, mono- or di-C₁-C₆alkylamino, mono- or di-alkylamide, C₁-C₂haloalkyl, and C₁-C₂haloalkoxy where any alkyl or alkoxy at R¹, R², R³, or R¹⁰ optionally has one methylene of the alkyl chain replaced by O, NH, N(C₁-C₄alkyl), or S and where the following compound is excluded:

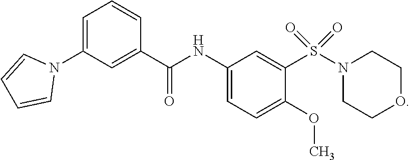 [SMILES: COc1ccc(NC(=O)c2cccc(-n3cccc3)c2)cc1S(=O)(=O)N1CCOCC1]

19. The method of claim 17, wherein the inhibition of eukaryotic translation initiation occurs in vivo in a patient administered a compound of claim 16 to the patient and the amount of the compound administered is an amount sufficient to provide a plasma concentration of the compound in that patient sufficient to reduce the interaction of eIF3e and eIF3d in vitro.

20. A method of modulating SIX1 and/or EYA2 levels or inhibiting SIX1/EYA2 interactions comprising contacting an eukaryotic translation initiation factor 3e (eIF3e) with a compound of the formula 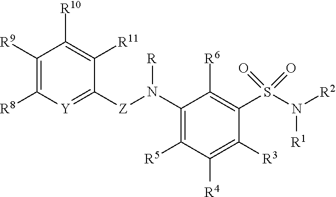
or a pharmaceutically acceptable salt thereof, wherein
Y is N or CR⁷;
Z is methylene (—CH₂—), keto (—(C═O)—), or is taken together with R⁵ to form a 5-membered partially unsaturated ring that contains 1 or 2 nitrogen atoms;
R is hydrogen or methyl;
R¹ is C₁-C₆alkyl (in which one CH₂ group is optionally replaced by O, NH, or S), or (C₃-C₆cyclopropyl)C₀-C₂alkyl; and R² is hydrogen or C₁-C₆alkyl; or
R¹ and R² are taken together to form a 5- or 6-membered heterocycloalkyl group optionally containing one additional heteroatom selected from N, O, and S, and optionally substituted with one or two substituents independently chosen from halogen, hydroxyl, C₁-C₄alkyl, C₁-C₄alkoxy, C₁-C₂haloalkyl, and C₁-C₂haloalkoxy;
R³ is halogen, hydroxyl, C₁-C₆alkyl, C₁-C₆alkoxy, C₁-C₂haloalkyl, C₁-C₂haloalkoxy, or (C₃-C₆cycloalkyl)C₀-C₂alkyl-O—;
R⁴, R⁶, R⁷, and R¹¹ are independently hydrogen, halogen, hydroxyl, C₁-C₄alkyl, C₁-C₄alkoxy, C₁-C₂haloalkyl, and C₁-C₂haloalkoxy;
R⁵ is hydrogen, halogen, hydroxyl, C₁-C₄alkyl, C₁-C₄alkoxy, C₁-C₂haloalkyl, or C₁-C₂haloalkoxy, or R⁵ is taken together with R⁵ to form a 5-membered partially unsaturated ring that contains 1 or 2 nitrogen atoms;
one of R⁸ and R⁹ is halogen, nitro, phenyl, di-C₁-C₄alkylamino, or a 4-6 membered saturated, partially unsaturated or aromatic heterocyclic group containing 1 or 2 additional heteroatoms selected from N, O, and S, each of which is optionally substituted with one or more substituents independently chosen from halogen, hydroxyl, cyano, amino, C₁-C₄alkyl, C₁-C₄alkoxy, C₁-C₂haloalkyl, and C₁-C₂haloalkoxy, and the other of R⁸ and R⁹ is hydrogen, halogen, hydroxyl, cyano, amino, C₁-C₂alkyl, C₁-C₂alkoxy, C₁-C₂haloalkyl, and C₁-C₂haloalkoxy;
R¹⁰ is hydrogen, halogen hydroxyl, cyano, amino, C₁-C₆alkyl, C₁-C₆alkoxy, mono- or di-C₁-C₆alkylamino, mono- or di-alkylamide, C₁-C₂haloalkyl, and C₁-C₂haloalkoxy, where any alkyl or alkoxy at R¹, R², R³, or R¹⁰ optionally has one methylene of the alkyl chain replaced by O, NH, N(C₁-C₄alkyl), or S and where the following compound is excluded:

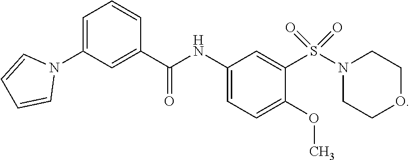 [SMILES: COc1ccc(NC(=O)c2cccc(-n3cccc3)c2)cc1S(=O)(=O)N1CCOCC1]

21. A method of treating metastatic breast cancer (of any subtype), glioblastoma, Wilms' tumor, ovarian cancer, lung cancer, cervical, oral cancer, colon cancer, lymphoma, osteosarcoma, or a breast cancer that is insensitive to hormone therapy, such as an ER⁺ tumors that are hormone resistant, in a patient comprising administering the patient a therapeutically effective amount of a compound of the formula 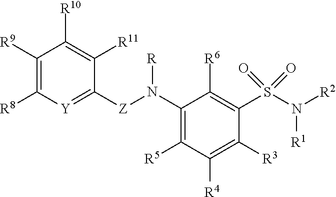
or a pharmaceutically acceptable salt thereof, wherein
Y is N or CR⁷;
Z is methylene (—CH₂—), keto (—(C═O)—), or is taken together with R⁵ to form a 5-membered partially unsaturated ring that contains 1 or two nitrogen atoms;
R is hydrogen or methyl;
R¹ is C₁-C₆alkyl or (C₃-C₆cyclopropyl)C₀-C₂alkyl; and
R² is hydrogen or C₁-C₆alkyl; or
R¹ and R² are taken together to form a 5- or 6-membered heterocycloalkyl group optionally containing one additional heteroatom selected from N, O, and S, and optionally substituted with one or two substituents independently chosen from halogen, hydroxyl, C₁-C₄alkyl, C₁-C₄alkoxy, C₁-C₂haloalkyl, and C₁-C₂haloalkoxy;
R³ is halogen, hydroxyl, C₁-C₆alkyl, C₁-C₆alkoxy, C₁-C₂haloalkyl, C₁-C₂haloalkoxy, or (C₃-C₆cycloalkyl)C₀-C₂alkyl-O—;
R⁴, R⁶, R⁷, and R¹¹ are independently hydrogen, halogen, hydroxyl, C₁-C₄alkyl, C₁-C₄alkoxy, C₁-C₂haloalkyl, and C₁-C₂haloalkoxy;
R⁵ is hydrogen, halogen, hydroxyl, C₁-C₄alkyl, C₁-C₄alkoxy, C₁-C₂haloalkyl, or C₁-C₂haloalkoxy, or is taken together with Z to form a 5-membered partially unsaturated ring that contains 1 or two nitrogen atoms;
one of R⁸ and R⁹ is halogen, nitro, phenyl, di-C₁-C₄alkylamino, or a 4-6 membered saturated, partially unsaturated or aromatic heterocyclic group containing 1 or 2 additional heteroatoms selected from N, O, and S, each of which is optionally substituted with one or more substituents independently chosen from halogen, hydroxyl, cyano, amino, C₁-C₄alkyl, C₁-C₄alkoxy, C₁-C₂haloalkyl, and C₁-C₂haloalkoxy, and the other of R⁸ and R⁹ is hydrogen, halogen, hydroxyl, cyano, amino, C₁-C₂alkyl, C₁-C₂alkoxy, C₁-C₂haloalkyl, and C₁-C₂haloalkoxy;
R¹⁰ is hydrogen, halogen hydroxyl, cyano, amino, C₁-C₆alkyl, C₁-C₆alkoxy, mono- or di-C₁-C₆alkylamino, mono- or di-alkylamide, C₁-C₂haloalkyl, and C₁-C₂haloalkoxy, where any alkyl or alkoxy at R¹, R², R³, or R¹⁰ optionally has one methylene of the alkyl chain replaced by O, NH, N(C₁-C₄alkyl), or S.

22. The method of claim 21, additionally comprising determining that the patient has a tumor or cancer in which eIF3e is elevated.

23. The method of claim 18, wherein the compound is a compound of the formula: 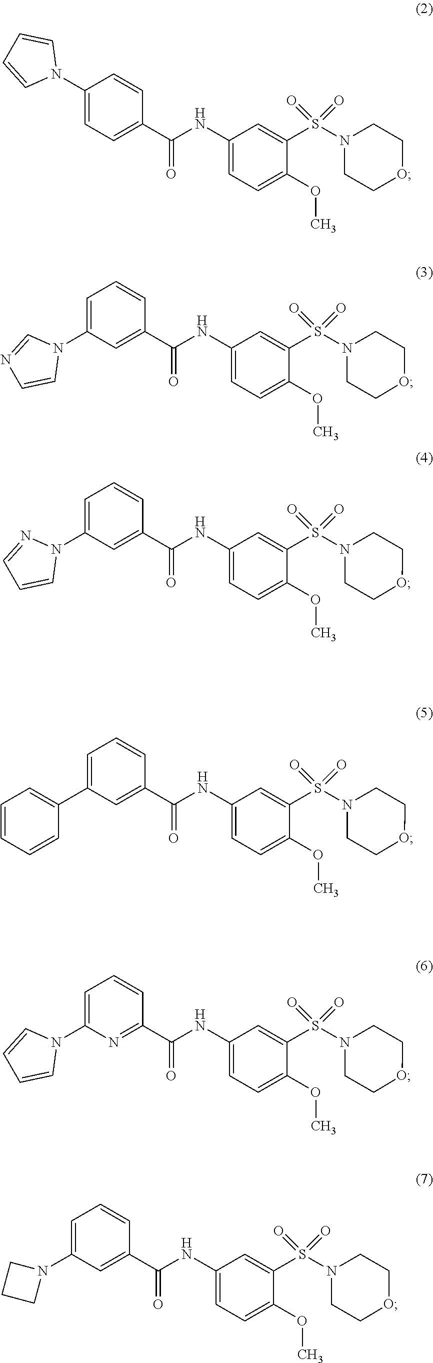 [SMILES: COc1ccc(NC(=O)c2ccc(-n3cccc3)cc2)cc1S(=O)(=O)N1CCOCC1 ; COc1ccc(NC(=O)c2cccc(-n3ccnc3)c2)cc1S(=O)(=O)N1CCOCC1 ; COc1ccc(NC(=O)c2cccc(-n3cccn3)c2)cc1S(=O)(=O)N1CCOCC1 ; COc1ccc(NC(=O)c2cccc(-c3ccccc3)c2)cc1S(=O)(=O)N1CCOCC1 ; COc1ccc(NC(=O)c2cccc(-n3cccc3)n2)cc1S(=O)(=O)N1CCOCC1 ; COc1ccc(NC(=O)c2cccc(N3CCC3)c2)cc1S(=O)(=O)N1CCOCC1] 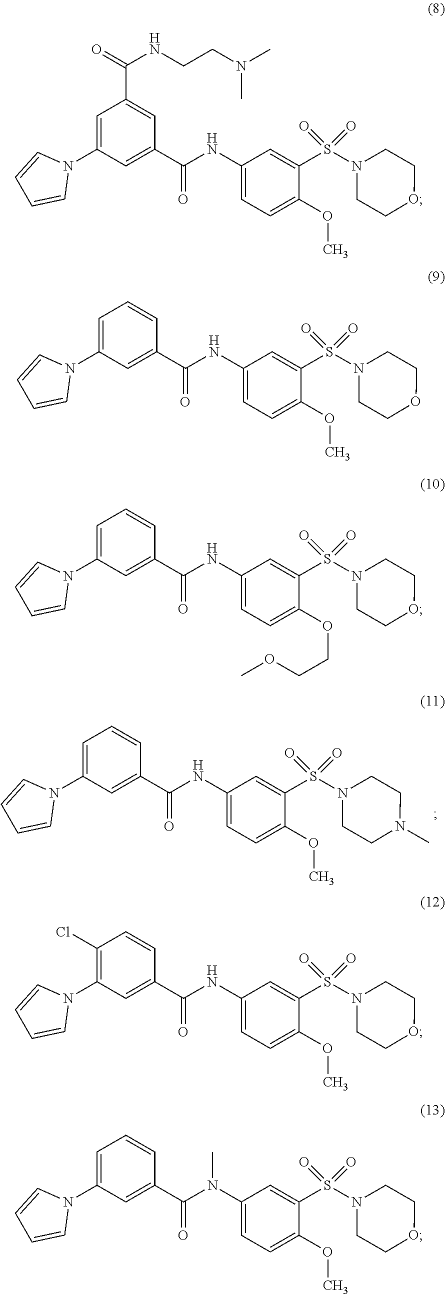 [SMILES: COc1ccc(NC(=O)c2cc(C(=O)NCCN(C)C)cc(-n3cccc3)c2)cc1S(=O)(=O)N1CCOCC1 ; COc1ccc(NC(=O)c2cccc(-n3cccc3)c2)cc1S(=O)(=O)N1CCOCC1 ; COCCOc1ccc(NC(=O)c2cccc(-n3cccc3)c2)cc1S(=O)(=O)N1CCOCC1 ; COc1ccc(NC(=O)c2cccc(-n3cccc3)c2)cc1S(=O)(=O)N1CCN(C)CC1 ; COc1ccc(NC(=O)c2ccc(Cl)c(-n3cccc3)c2)cc1S(=O)(=O)N1CCOCC1 ; COc1ccc(N(C)C(=O)c2cccc(-n3cccc3)c2)cc1S(=O)(=O)N1CCOCC1] 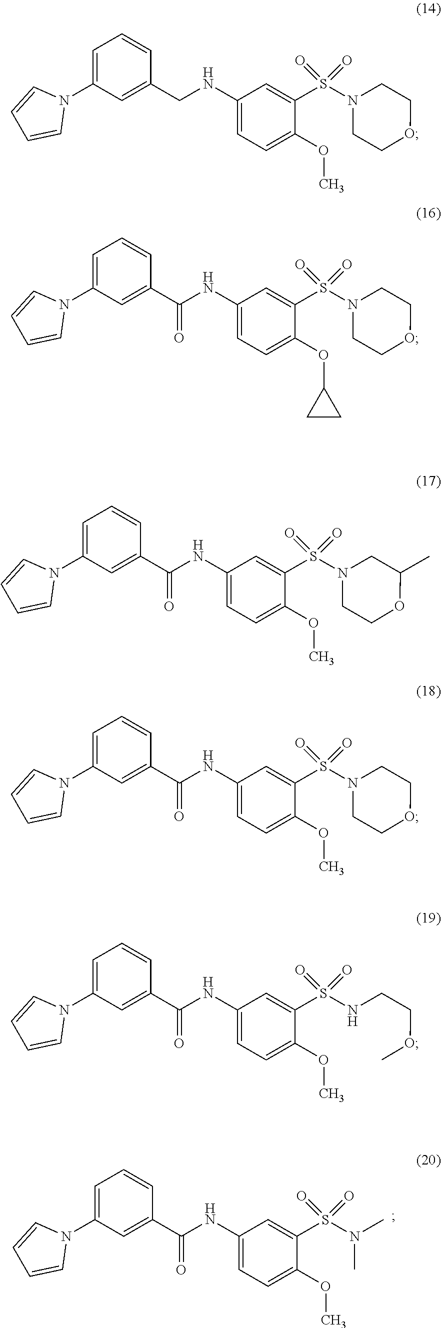 [SMILES: COc1ccc(NCc2cccc(-n3cccc3)c2)cc1S(=O)(=O)N1CCOCC1 ; O=C(Nc1ccc(OC2CC2)c(S(=O)(=O)N2CCOCC2)c1)c1cccc(-n2cccc2)c1 ; COc1ccc(NC(=O)c2cccc(-n3cccc3)c2)cc1S(=O)(=O)N1CCOC(C)C1 ; COc1ccc(NC(=O)c2cccc(-n3cccc3)c2)cc1S(=O)(=O)N1CCOCC1 ; COCCNS(=O)(=O)c1cc(NC(=O)c2cccc(-n3cccc3)c2)ccc1OC ; COc1ccc(NC(=O)c2cccc(-n3cccc3)c2)cc1S(=O)(=O)N(C)C] 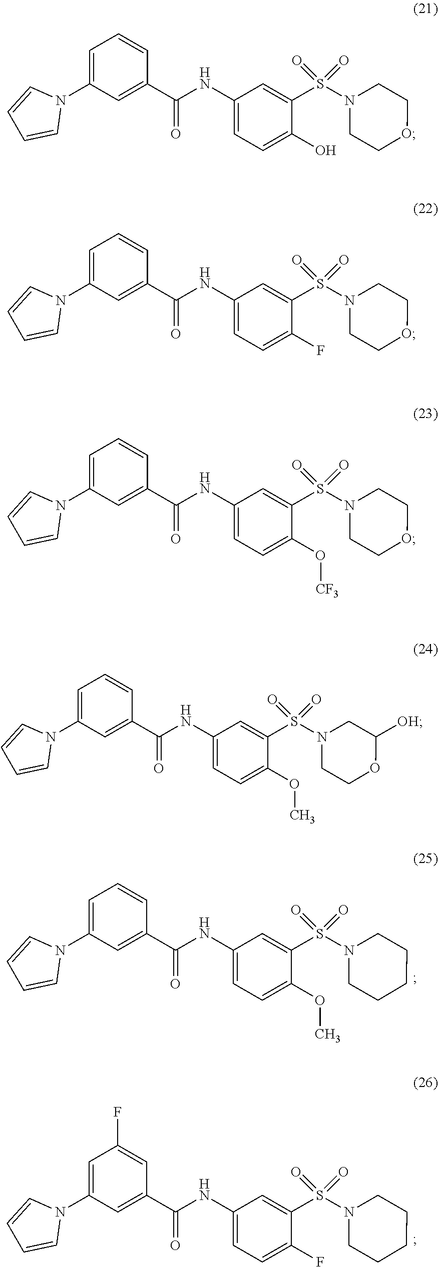 [SMILES: O=C(Nc1ccc(O)c(S(=O)(=O)N2CCOCC2)c1)c1cccc(-n2cccc2)c1 ; O=C(Nc1ccc(F)c(S(=O)(=O)N2CCOCC2)c1)c1cccc(-n2cccc2)c1 ; O=C(Nc1ccc(OC(F)(F)F)c(S(=O)(=O)N2CCOCC2)c1)c1cccc(-n2cccc2)c1 ; COc1ccc(NC(=O)c2cccc(-n3cccc3)c2)cc1S(=O)(=O)N1CCOC(O)C1 ; COc1ccc(NC(=O)c2cccc(-n3cccc3)c2)cc1S(=O)(=O)N1CCCCC1 ; O=C(Nc1ccc(F)c(S(=O)(=O)N2CCCCC2)c1)c1cc(F)cc(-n2cccc2)c1] 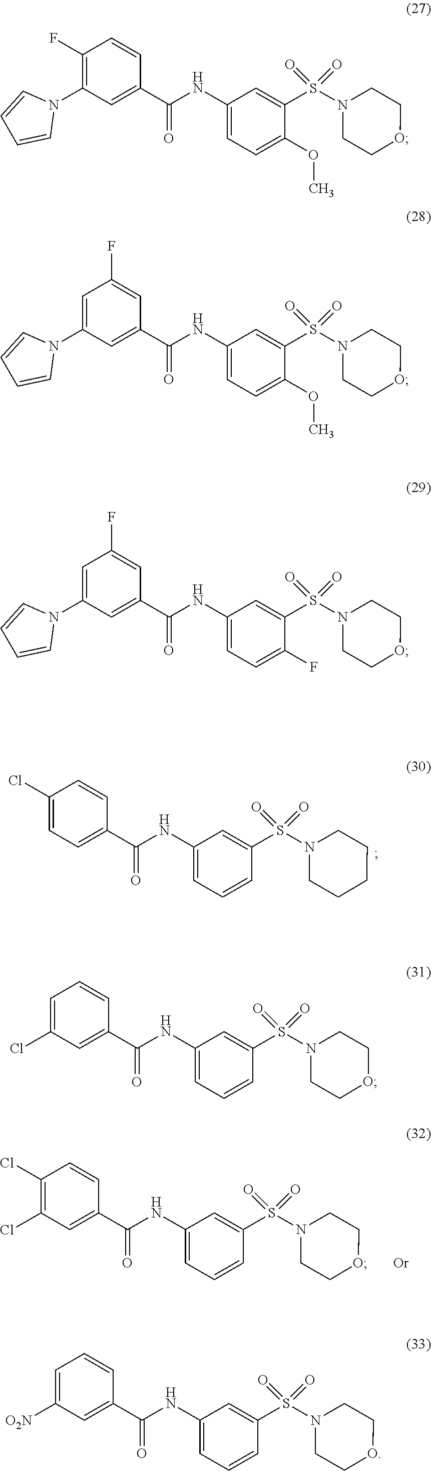
or a pharmaceutically acceptable salt of any of the foregoing.

24. A method of reducing or inhibiting translation of an mRNA, where the translation of the mRNA is initiated by eIF3e, and the mRNA encodes a protein linked to cell proliferation, apoptosis, cell migration/invasion, cellular respiration, cellular stress response, or cell differentiation, comprising contacting the eIF3e with a compound or salt of claim 1, wherein the mRNA is an mRNA encodes SIX1. EYA2, c-Jun, or HIF1.

25. (canceled)
